(19) United States
(12) Patent Application Publication
(10) Pub. No.: US 2026/0043069 A1
(43) Pub. Date: Feb. 12, 2026

(54) METHODS, SYSTEMS AND COMPOSITIONS FOR ANALYTE DETECTION

(71) Applicant: Cleancard Inc., San Francisco, CA (US)

(72) Inventors: Thomas M. CARROLL, San Francisco, CA (US); Luca Floyd River Merlin Springer, San Francisco, CA (US); Dominique Piché, Los Altos, CA (US); Chandler Abraham, San Francisco, CA (US); Liam Walmsley-Eyre, Aviemore (GB); Hari K. K. Subramanian, San Mateo, CA (US)

(21) Appl. No.: 19/353,137

(22) Filed: Oct. 8, 2025

Related U.S. Application Data
(63) Continuation of application No. PCT/US2025/041357, filed on Aug. 8, 2025.
(60) Provisional application No. 63/681,640, filed on Aug. 9, 2024.
(60) Provisional application No. 63/729,836, filed on Dec. 9, 2024.

(51) Int. Cl. C12Q 1/6809; C12N 9/22; C12N 11/00; C12N 15/113; C12Q 1/34

(52) U.S. Cl. CPC C12Q 1/6809; C12N 9/222; C12N 11/00; C12N 15/113; C12Q 1/34; C12N 2310/16; C12N 2310/20

(57) ABSTRACT

Provided herein are methods, systems, and compositions for detecting and/or quantifying analytes. The methods, systems and compositions may comprise a first component of a signal-generating complex and a second component of signal-generating complex. The first component may be immobilized to a solid substrate via a disruptable linker at a first location of a reaction vessel and a second component may be immobilized to the solid substrate at a second location of the reaction vessel. The linker may be disrupted allowing the signal-generating complex to form.

### CROSS-REFERENCE

This application is a continuation of International Application No. PCT/US2025/041357, filed Aug. 8, 2025 which claims the benefit of U.S. Application No. 63/681,640, filed Aug. 9, 2024, and U.S. Application No. 63/729,836, filed Dec. 9, 2024, each of which is incorporated by reference herein in its entirety.

### BACKGROUND

Detection of biomarkers in individuals may be useful for identifying diseases or disorders in a subject. Disorders may be caused by genetic aberrations or aberrant expression of polypeptides. The use of assays for detection of analytes may provide information about the status of a subject.

### SUMMARY

Provided herein are methods, systems, and compositions for detection of analytes. The methods and systems may use separated components of a signal-generating complex to generate a signal based at least in part on the presence of an analyte.

In an aspect, the present disclosure provides a method of generating a signal indicative of a presence of an analyte, the method comprising: (a) providing a solid substrate comprising: (i) a first component of a signal-generating complex, wherein the first component is immobilized to the solid substrate via a disruptable linker at a first location of a reaction vessel, and (ii) a second component of the signal-generating complex, wherein the second component is immobilized to the solid substrate at a second location of the reaction vessel; (b) contacting the sample with the solid substrate, wherein the linker is disrupted based at least in part on a presence of the analyte, thereby releasing the first component; and (c) associating the first component to the second component to form a functional signal-generating complex immobilized to the solid substrate, wherein the functional signal-generating complex generates a signal indicative of a presence or quantity of the analyte in the sample.

In some embodiments, the first component comprises an enzyme. In some embodiments, the first component comprises an apoenzyme. In some embodiments, the first component comprises a peroxidase. In some embodiments, the peroxidase comprises a horseradish peroxidase. In some embodiments, the peroxidase comprises a guanine-rich deoxyribozyme (DNAzyme). In some embodiments, the second component comprises a cofactor. In some embodiments, the cofactor comprises a porphyrin. In some embodiments, the porphyrin is a hemin. In some embodiments, the hemin a modified hemin. In some embodiments, the functional signal-generating complex comprises a holoenzyme.

In some embodiments, the method further comprises, subsequent to (c), incorporating a pro-signal molecule into the functional signal-generating complex and processing the pro-signal molecule to generate the signal. In some embodiments, the pro-signal molecule is colorless. In some embodiments, the pro-signal molecule comprises TMB (3,3′,5,5′-tetramethylbenzidine). In some embodiments, the pro-signal molecule comprises ABTS (2,2′-azino-bis(3-ethylbenzothiazoline-6-sulfonic acid)), a resazurin derivative (e.g., 10-acetyl-3,7-dihydroxyphenoxazine), or luminol. In some embodiments, the signal comprises a colorimetric signal. In some embodiments, the signal comprises a fluorometric signal. In some embodiments, the signal comprises an electrochemical signal. In some embodiments, the signal comprises a luminescent signal. In some embodiments, the disruptable linker comprises a ribonucleic acid (RNA). In some embodiments, the disruptable linker comprises a deoxyribonucleic acid (DNA). In some embodiments, the disruptable linker comprises a polypeptide. In some embodiments, the disruptable linker comprises a small molecule (e.g., saccharide), and the analyte displaces the small molecule from an immobilized binder. In some embodiments, the disruptable linker comprises a substrate for a catabolic enzyme. In some embodiments, the analyte comprises the catabolic enzyme. In some embodiments, the disruptable linker is configured to be cleaved by the analyte.

In some embodiments, the disruptable linker comprises a restriction enzyme recognition site. In some embodiments, the disruptable linker comprises one or more disruptable regions. In some embodiments, the one or more disruptable regions comprise a first disruptable region capable of being cleaved by a sequence-specific restriction enzyme and a second disruptable region capable of being cleaved by a nucleotide-specific enzyme. In some embodiments, the one or more disruptable regions comprise double-stranded deoxyribonucleic acid (dsDNA). In some embodiments, the disruptable linker is disrupted via strand displacement. In some embodiments, the disruptable linker is disrupted by a DNA or RNA-cleaving DNAzyme.

In some embodiments, the method further comprises, prior to b), contacting the solid substrate with a cleaving agent. In some embodiments, the cleaving agent comprises a CRISPR-associated system (Cas) protein. In some embodiments, the Cas protein comprises a Cas13 protein. In some embodiments, the Cas protein comprises a Cas 12 protein. In some embodiments, the cleaving reagent comprises a CRISPR/Cas complex. In some embodiments, the CRISPR/Cas complex comprises a guide RNA, wherein the guide RNA is complementary to the analyte. In some embodiments, the disruptable linker comprises a self-cleaving linker. In some embodiments, the self-cleavable linker (e.g., DNA self-cleavable linker) comprises an oligonucleotide aptamer. In some embodiments, the oligonucleotide aptamer comprises DNA. In some embodiments, the oligonucleotide aptamer comprises RNA. In some embodiments, the oligonucleotide aptamer comprises DNA and RNA. In some embodiments, the oligonucleotide aptamer is configured to bind to the analyte, and wherein the self-cleavable linker is configured to be cleaved upon binding to the analyte. In some embodiments, the self-cleavable linker is cleaved at an RNA base of the oligomer aptamer.

In some embodiments, the solid substrate further comprises a signal-amplifying complex, wherein the signal-amplifying complex comprises a catalytic agent immobilized to the solid substrate via a second disruptable linker. In some embodiments, the signal-amplifying complex further comprises an analyte binding module capable of binding to the analyte. In some embodiments, the second disruptable linker is disrupted upon binding of the analyte to the analyte-binding module. In some embodiments, (b) further comprises (i) binding the analyte to the analyte-binding module, thereby releasing the catalytic agent, and (ii) cleaving, by the catalytic agent, the disruptable linker, thereby releasing the first component of a signal-generating complex. In some embodiments, the disruptable linker comprises a substrate of the catalytic agent. In some embodiments, the catalytic agent comprises a restriction enzyme. In some embodiments, the second disruptable linker further comprises biotin, wherein the biotin interacts with the solid substrate. In some embodiments, the first location comprises a plurality of first components of signal-generating complexes. In some embodiments, the plurality of first components is immobilized via a plurality of disruptable linkers. In some embodiments, the first location comprises a plurality of first components of signal-generating complexes, wherein the plurality of first components is immobilized via a plurality of disruptable linkers, and wherein the catalytic agent disrupts the plurality of disruptable linkers, thereby releasing the plurality of first components. In some embodiments, the solid substrate further comprises a second signal-amplifying complex, wherein the second signal-amplifying complex comprises a second catalytic agent immobilized to the solid substrate via a third disruptable linker. In some embodiments, the second disruptable linker comprises a substrate for the second catalytic agent. In some embodiments, the second signal-amplifying complex comprises an analyte-binding module, and wherein (b) further comprises binding the analyte to the analyte-binding module, thereby releasing the second catalytic agent. In some embodiments, (b) further comprises (i) cleaving, by the second catalytic agent, the second disruptable linker thereby releasing the catalytic agent, and (ii) allowing the catalytic agent to cleave the disruptable linker, thereby releasing the first component.

In some embodiments, the analyte comprises a ribonucleic acid (RNA). In some embodiments, the analyte comprises a deoxyribonucleic acid (DNA). In some embodiments, the analyte comprises a polypeptide. In some embodiments, the analyte comprises a saccharide (e.g., hyaluronic acid or glycan). In some embodiments, the analyte comprises an enzyme. In some embodiments, the sample comprises a cell. In some embodiments, the sample is derived from a tissue. In some embodiments, the sample is derived from a bodily fluid. In some embodiments, the bodily fluid comprises blood, plasma, urine, semen, or saliva, sweat, or mucus. In some embodiments, the sample is derived from stool or fecal matter. In some embodiments, the reaction vessel comprises a well. In some embodiments, the well is a microwell.

In some embodiments, the method comprises, subsequent to (c), using a detector to detect the signal indicative of the presence of the analyte in the sample. In some embodiments, the detector comprises a spectrophotometer. In some embodiments, the detector comprises a fluorometer. In some embodiments, the detector comprises a luminometer. In some embodiments, the detector comprises a potentiostat. In some embodiments, the detector comprises a plate reader. In some embodiments, the detector comprises an imaging device. In some embodiments, the imaging device comprises a camera. In some embodiments, the camera is a component of a mobile device. In some embodiments, the signal comprises a colorimetric signal, and wherein the detector is configured to detect the colorimetric signal. In some embodiments, the signal comprises a fluorescent signal, and wherein the detector is configured to detect the fluorescent signal. In some embodiments, the signal comprises an electrochemical signal, and wherein the detector is configured to detect the electrochemical signal. In some embodiments, the signal comprises a luminescent signal, and wherein the detector is configured to detect the luminescent signal.

In another aspect, the present disclosure provides a method of detecting an analyte as present in at least a concentration in a single reaction vessel, wherein the detecting is performed with a sensitivity of at least 90%, a specificity of at least 90%, and within a time of no greater than 30 minutes. In some embodiments, the threshold concentration comprises at least 10 pM (picomolar). In some embodiments, the threshold concentration comprises at least 100 pM. In some embodiments, the detecting is performed within a time of no greater than 15 minutes. In some embodiments, the detecting is performed within a time of no greater than 5 minutes. In some embodiments, the detecting is performed with a sensitivity of at least 95%. In some embodiments, the detecting is performed with a sensitivity of at least 99%. In some embodiments, the detecting is performed with a specificity of at least 95%. In some embodiments, the detecting is performed with a specificity of at least 99%.

In another aspect, the present disclosure provides a system for detecting an analyte in a sample, comprising: (a) a solid substrate comprising: (i) a first component of a signal-generating complex, wherein the first component is immobilized to the solid substrate via a disruptable linker at a first location of a reaction vessel, and (ii) a second component of the signal-generating complex, wherein the second component is immobilized to the solid substrate at a second location of the reaction vessel; wherein the disruptable linker is cleaved based on a presence of the analyte, upon contacting the sample with the solid substrate thereby releasing the first component; wherein a functional signal-generating complex is formed upon associating the first component to the second component, wherein a signal is generated from the functional signal-generating complex; and (b) a detector configured to detect the signal, wherein the detected signal is indicative of a presence of the analyte in the sample.

In some embodiments, the first component comprises an enzyme. In some embodiments, the first component comprises an apoenzyme. In some embodiments, the first component comprises a peroxidase. In some embodiments, the peroxidase comprises a horseradish peroxidase. In some embodiments, the second component comprises a cofactor. In some embodiments, the cofactor comprises a hemin. In some embodiments, the functional signal-generating complex comprises a holoenzyme. In some embodiments, the functional signal-generating complex is configured to process a pro-signal molecule to generate the signal. In some embodiments, the pro-signal molecule is colorless. In some embodiments, the pro-signal molecule comprises TMB (3,3′,5,5′-tetramethylbenzidine) In some embodiments, the pro-signal molecule comprises ABTS (2,2′-azino-bis(3-ethylbenzothiazoline-6-sulfonic acid)). In some embodiments, the signal comprises a colorimetric signal. In some embodiments, the signal comprises a fluorometric signal. In some embodiments, the signal comprises an electrochemical signal. In some embodiments, the signal comprises a luminescent signal.

In some embodiments, the disruptable linker comprises a ribonucleic acid (RNA). In some embodiments, the disruptable linker comprises a deoxyribonucleic acid (DNA). In some embodiments, the disruptable linker comprises a polypeptide. In some embodiments, the disruptable linker comprises a substrate for a catabolic enzyme. In some embodiments, the analyte comprises the catabolic enzyme. In some embodiments, the disruptable linker comprises a small molecule (e.g., saccharide). In some embodiments, the disruptable linker is configured to be released from a binder in response to the analyte. In some embodiments, the disruptable linker is configured to be cleaved by the analyte. In some embodiments, the disruptable linker comprises a restriction enzyme recognition site. In some embodiments, the disruptable linker comprises one or more disruptable regions. In some embodiments, the one or more disruptable regions comprise a first disruptable region capable of being cleaved by a sequence-specific restriction enzyme and a second disruptable region capable of being cleaved by a nucleotide-specific enzyme. In some embodiments, the one or more disruptable regions comprise double-stranded deoxyribonucleic acid (dsDNA).

In some embodiments, the disruptable linker is configured to be disrupted via strand displacement. In some embodiments, the disruptable linker is disrupted by a DNA or RNA-cleaving DNAzyme. In some embodiments, the disruptable linker is configured to be cleaved by a cleaving agent. In some embodiments, the cleaving agent comprises a CRISPR-associated system (Cas) protein. In some embodiments, the Cas protein comprises a Cas13 protein. In some embodiments, the Cas protein comprises a Cas12 protein. In some embodiments, the cleaving reagent comprises a CRISPR/Cas complex. In some embodiments, the CRISPR/Cas complex comprises a guide RNA, wherein the guide RNA is complementary to the analyte. In some embodiments, the disruptable linker comprises a self-cleaving linker. In some embodiments, the self-cleavable linker comprises an oligonucleotide aptamer. In some embodiments, the oligonucleotide aptamer comprises DNA. In some embodiments, the oligonucleotide aptamer comprises RNA. In some embodiments, the oligonucleotide aptamer comprises DNA and RNA. In some embodiments, the oligonucleotide aptamer is configured to bind to the analyte, and wherein the self-cleavable linker is configured to be cleaved upon binding to the analyte. In some embodiments, the self-cleavable linker is cleaved at an RNA base of the oligomer aptamer.

In some embodiments, the solid substrate further comprises a signal-amplifying complex, wherein the signal-amplifying complex comprises a catalytic agent immobilized to the solid substrate via a second disruptable linker. In some embodiments, the signal-amplifying complex further comprises an analyte binding module capable of binding to the analyte. In some embodiments, the second disruptable linker is disrupted upon binding of the analyte to the analyte-binding module. In some embodiments, the signal-amplifying complex is configured to bind the analyte to the analyte-binding module, thereby releasing the catalytic agent, and wherein the catalytic agent is configured to cleave the disruptable linker, thereby releasing the first component. In some embodiments, the disruptable linker comprises a substrate of the catalytic agent. In some embodiments, the catalytic agent comprises a restriction enzyme. In some embodiments, the second disruptable linker further comprises biotin, wherein the biotin interacts with the solid substrate. In some embodiments, the first location comprises a plurality of first components of signal-generating complexes. In some embodiments, the plurality of first components is immobilized via a plurality of disruptable linkers. In some embodiments, the first location comprises a plurality of first components of signal-generating complexes, wherein the plurality of first components is immobilized via a plurality of disruptable linkers, and wherein the catalytic agent disrupts the plurality of disruptable linkers, thereby releasing the plurality of first components. In some embodiments, the solid substrate further comprises a second signal-amplifying complex, wherein the second signal-amplifying complex comprises a second catalytic agent immobilized to the solid substrate via a third disruptable linker. In some embodiments, the second disruptable linker comprises a substrate for the second catalytic agent. In some embodiments, the second signal-amplifying complex comprises an analyte-binding module, and the second signal-amplifying complex is configured to bind the analyte to the analyte-binding module to release the second catalytic agent. In some embodiments, the second catalytic agent is configured to cleave the second disruptable linker and release the catalytic agent, and the catalytic agent is configured to cleave the disruptable linker to release the first component.

In some embodiments, the analyte comprises a ribonucleic acid (RNA). In some embodiments, the analyte comprises a deoxyribonucleic acid (DNA). In some embodiments, the analyte comprises a saccharide. In some embodiments, the analyte comprises a polypeptide. In some embodiments, the analyte comprises an enzyme. In some embodiments, the sample comprise a cell. In some embodiments, the sample is derived from a tissue. In some embodiments, the sample is derived from a bodily fluid. In some embodiments, the bodily fluid comprises blood, plasma, urine, saliva, sweat, or mucus. In some embodiments, the sample is derived from stool or fecal matter. In some embodiments, the reaction vessel comprises a well. In some embodiments, the well is a microwell. In some embodiments, the detector comprises a spectrophotometer. In some embodiments, the detector comprises a fluorometer. In some embodiments, the detector comprises a luminometer. In some embodiments, the detector comprises a potentiostat. In some embodiments, the detector comprises a plate reader. In some embodiments, the detector comprises an imaging device. In some embodiments, the imaging device comprises a camera. In some embodiments, the camera is a component of a mobile device. In some embodiments, the detector is configured to detect a colorimetric signal. In some embodiments, the detector is configured to detect a fluorescent signal. In some embodiments, the detector is configured to detect a luminescent signal. In some embodiments, the detector is configured to detect an electrochemical signal.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure”and “FIG.”herein), of which:

FIGS. 1A-1B show a schematic of an example system of the present disclosure.

FIG. 2 shows an example CRISPR-associated nuclease construct for use in the systems and methods of the present disclosure.

FIG. 3 shows an example oligonucleotide aptamer construct for use in the systems and methods of the present disclosure.

FIGS. 4A-4B show an example schematics of signal amplification complexes and their use in the systems and methods of the present disclosure.

FIG. 5 shows a computer system that is programmed or otherwise configured to implement a method of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are systems and methods for detecting analytes. The methods may be used to assay for the presence or absence of an analyte in a sample derived from a subject. Detection of analytes from a subject may allow for the detection of a specific biomarker in a subject and may allow for a disease or condition to be identified as present in a subject. For example, detection of a particular nucleotide sequence may indicate the expression of a particular gene in a subject.

The methods, systems, and compositions described in the present disclosure may comprise the use of a multi-component signal-generating complex that can generate a signal when the components are combined or joined together. At an initial state, the components may be immobilized or sequestered such that the components are unable to combine, thus preventing the generation of a signal. The components may be mobilized and allowed to combine, and thus may be able to generate the signal. As such, by disrupting the immobilizing features, the previously immobilized components may be able to react and generate a signal. By making the activation of the component mobilization dependent upon the presence of an analyte, the analyte can be detected based at least in part on the signal that is generated.

The methods, systems, and compositions described in the present disclosure may be advantageous over other analyte detection methods by allowing for the reaction to take place in a single reaction vessel. The methods, systems and compositions may also allow for fewer washing or rinsing operations than may be required in conventional assays. The methods, systems, and compositions may also allow for greater signal amplification than is feasible in conventional rapid testing approaches. The methods, systems, and compositions may be more sensitive, more specific, more accurate, or faster than conventional assays. The methods may also allow for a greater degree of multiplexing of reactions within a single device, than is feasible in conventional assays. The methods, systems, composition may also allow for the production of devices or assays that are easier to use or implement by a lay person or non-professional user, than is feasible using conventional methods. For example, the methods, systems, and compositions may allow for assays that can be performed outside of a laboratory setting, such as a subject's home, and can still maintain a high level of accuracy, sensitivity, and/or specificity.

In an aspect, the present disclosure provides a method of detecting an analyte in a sample, the method comprising: (a) providing a solid substrate comprising: (i) a first component of a signal-generating complex, wherein the first component is immobilized to the solid substrate via a disruptable linker at a first location of a reaction vessel, and (ii) a second component of the signal-generating complex, wherein the second component is immobilized to the solid substrate at a second location of the reaction vessel; (b) contacting the sample with the solid substrate, wherein the linker is disrupted in a presence of the analyte, thereby releasing the first component; (c) associating the first component to the second component to form a functional signal-generating complex immobilized to the solid substrate, which functional signal-generating complex generates a signal indicative of a presence of the analyte in the sample; and (d) using a detector to detect the signal indicative of the presence of the analyte in the sample.

In another aspect, the present disclosure provides compositions and systems for the use of methods of the present disclosure. In an aspect, the present disclosure provides a system for detecting an analyte in a sample, comprising: a solid substrate comprising: (i) a first component of a signal-generating complex, wherein the first component is immobilized to the solid substrate via a disruptable linker at a first location of a reaction vessel, and (ii) a second component of the signal-generating complex, wherein the second component is immobilized to the solid substrate at a second location of the reaction vessel. The system may comprise a disruptable linker that is disrupted based at least in part on a presence of an analyte. The disruptable linker may be disrupted upon contacting the sample with the solid substrate and can release the first component. The system may allow the functional signal-generating complex to be formed upon associating the first component to the second component, wherein a signal is generated from the functional signal-generating complex. The system may comprise a detector configured to detect a signal. The detected signal can be indicative of a presence of the analyte in the sample.

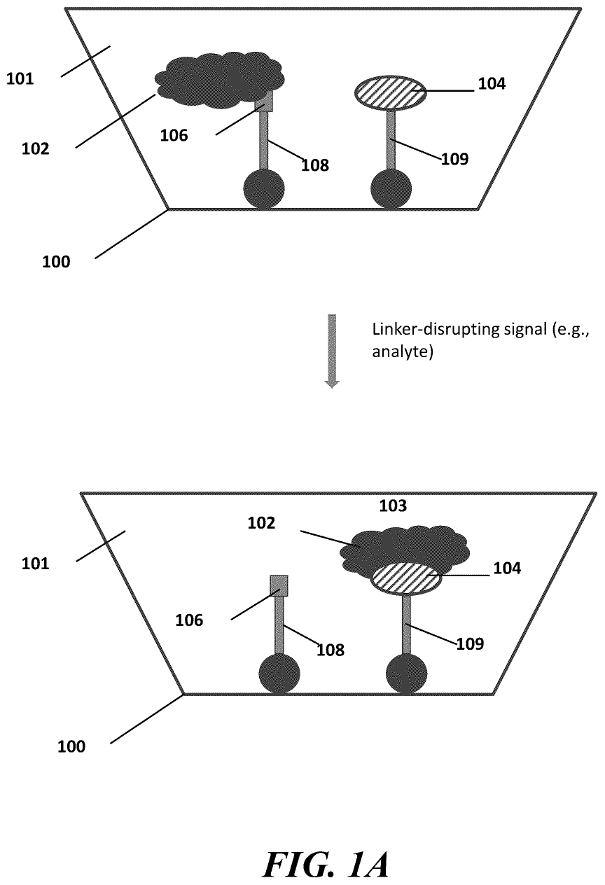

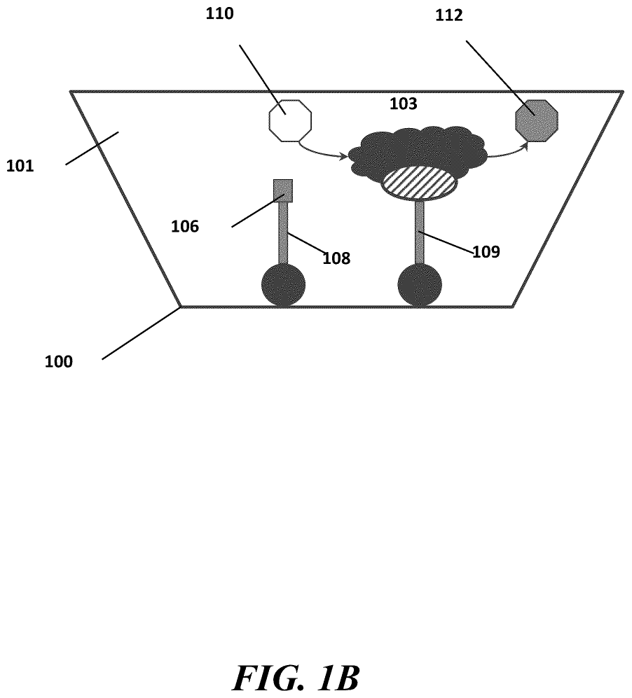

FIGS. 1A-1B show a schematic of an example assay. As shown in the top schematic of FIG. 1A, a first component 102 (e.g., an apoperoxidase) may be initially immobilized to a solid substrate 100 by the use of a linker 108. The linker may be disruptable such that upon a stimulus (e.g., presence of the analyte), the linker can release the first component from the solid substrate. A disruptable section of the linker 106 can be cleaved or allowed to dissociate to allow the first component 102 to be released. The second component 104 (e.g., hemin, porphyrin ring, or other porphyrin compound) may be immobilized in a same reaction vessel and may be at a second location and be unable to interact with the first component. The second component 104 may be immobilized to the solid substrate 100 by a linker 109. This linker 109 may be a stable or non-disruptable linker, such that the stimulus that disrupts the disruptable linker does not affect the stable linker. Upon addition of a linker disrupting signal, such as the presence of the analyte, the disruptable linker section 106 can be disrupted, and the first component 102 can be released. As shown in the bottom schematic of FIG. 1A, the second component 104 and first component 102 can interact form a signal-generating complex 103 (e.g., holoperoxidase), which is able to generate a signal.

FIG. 1B shows a schematic of the example assay after a signal-generating complex has been formed. The first component is no longer immobilized by linker 108 and has formed signal-generating complex 103 which can be immobilized to the solid substrate via linker 109. A pro-signal molecule 110 can be added to the reaction vessel 101. The signal-generating complex 103 can react with pro-signal molecule 110 and generate a signal molecule 112. Signal molecule 112 can then be detected by a detector.

As shown, the signal-generating complex can be formed by releasing a first component of a signal-generating complex and allowing it to interact with a second component of the signal-generating complex. As such, making a release of the first component be dependent on or activated by the presence of an analyte may allow the system to detect an analyte. The provided disclosure demonstrates methods, compositions, and systems for activating the formation of the signal-generating complex based at least in part on a presence of an analyte.

The methods, compositions, and systems of the present disclosure may use various analyte detection modules for detection of various types of analytes. For example, RNA analytes, DNA analytes, polypeptide analytes, and saccharide analytes (e.g., polysaccharides, such as glycans and hyaluronic acid) may be detected using various systems that disrupt the linkers and may use a similar signal-generating complex and pro-signal molecules, to generate the signal. As such, various signal-generating complexes, linkers, and cleaving and/or dissociating reagents may be used to detect an analyte.

### Signal-Generating Complexes

The signal-generating complex may comprise various components to make a functional complex. A component of the signal-generating complex may comprise an enzyme. For example, the enzyme may modify a substrate such to generate a signal. For example, the enzyme may react with a colorless substrate, that upon an enzymatic reaction, generates a substrate with color. For example, the enzyme may be a peroxidase. Peroxidases may be enzymes that are able to break up peroxides. These peroxidases can catalyze a reaction with a peroxide by performing redox chemistry on the peroxides. The catalysis may use specific substrates as electron donors or acceptors that upon reaction may transform a compound or molecule into a compound or molecule that is more easily detectable. For example, a substrate may be a colorless substrate and may be transformed into a molecule that has a color. The peroxidase may be a horseradish peroxidase (HRP). The peroxidase may be a guanine-rich DNAzyme. The peroxidase (e.g., HRP or guanine-rich DNAzyme) may react with a variety of substrates to generate molecules that comprise a color. For example, the peroxidase (e.g., HRP or guanine-rich DNAzyme) can react with ABTS, TMB, luminol, or other chromogenic substrates, or chemiluminescent substrates. The substrates upon reaction (e.g., oxidation) with the peroxidase (e.g., HRP or guanine-rich DNAzyme) may produce substrates that are colored or produce luminescence. This color or luminescence can be detected by an instrument or the naked eye such to identify that a functional enzyme (e.g., HRP, guanine-rich DNAzyme, or other peroxidase) is present. When linked to other reactions, as described throughout the present disclosure, the presence of the functional enzyme can be correlated to or indicative of the presence of an analyte.

The component of the signal-generating complex may be an apoenzyme. As described, the component may be an enzyme that can catalyze a reaction to produce a signal. An apoenzyme can refer to an enzyme that is missing a cofactor of the enzyme. Without the cofactor, the enzyme may be inactive or unable to catalyze a reaction. An apoenzyme may be activated by contacting with the cofactor, thereby generating an active holoenzyme. The activation of the apoenzyme by binding of a cofactor may be used to modulate the activity of the enzyme. The addition or availability of the cofactor may be dependent on the presence of an analyte. As such, the enzyme may be inactive (and unable to generate a signal) when an analyte is absent, and the enzyme may be active when an analyte is present. Peroxidases may need the presence of a cofactor to be active. An apo-peroxidase (e.g., apo-HRP) may be a first component of a signal-generating complex. Upon addition of the cofactor, the peroxidase may be active and generate a signal.

A component of the signal-generating complex may be a cofactor. The cofactor may allow an enzyme to function properly, for example, an enzyme may be active with a cofactor or inactive without a cofactor. The cofactor may comprise an ion. For example. the cofactor may comprise a metal or a metal ion. The cofactor may comprise iron, magnesium, manganese, cobalt, copper, zinc, or molybdenum. The cofactor may comprise an organic compound. For example, the cofactor may comprise a porphyrin ring. The porphyrin ring may be complexed to a metal ion. For example, the cofactor may comprise a hemin. The hemin may allow the enzyme (e.g., a peroxidase) to perform a redox reaction. In some case, the cofactor may comprise a modified or non-native cofactor. For example, the hemin may be modified hemin.

As described in the present disclosure, the functional signal-generating complex may comprise multiple components. The functional signal-generating complex may comprise a holoenzyme. A first component of the signal-generating complex may comprise an apoenzyme, and a second component of the signal-generating complex may comprise a cofactor. The binding of the apoenzyme to the cofactor may generate a holoenzyme that is active and can generate a signal by catalyzing a reaction.

As described in the present disclosure, the components of signal-generating complexes may be immobilized to a solid substrate at a location separate from another component. Multiple copies of components (e.g., a first component of a signal-generating complex or a second component of a signal-generating complex) may be immobilized for a given assay. For example, a first location of solid substrate may comprise a plurality of first components immobilized via a plurality of disruptable linkers. Similarly, a second location of solid substrate may comprise a plurality of second components of a signal-generating complex immobilized to the solid substrate.

### Signal Amplification Complexes

The methods and compositions of the present disclosure may comprise signal amplification complexes. Signal amplification complexes may allow for the signals to be greater in intensity or strength as compared to another assay that does not comprise signal amplification complexes. As described throughout the present disclosure, an analyte may be present in a sample and may cause a signal to be generated via a signal-generating complex. For example, as described elsewhere herein, an analyte may directly cleave or may bind to a self-cleaving aptamer to cleave, a disruptable linker and release a first component of a signal-generating complex which can form a complete signal-generating complex and generate a signal. In this case, the stochiometric ratio of the analyte to released first components of the signal-generating complex may be close to 1:1. As such, the signal generation may have a linear correlation (or close to linear correlation) with signal generation and may generate a small signal intensity in the presence of a small amount of analyte. This may result in a less sensitive assay and may make detection of a small amount of analyte more difficult. A signal amplification complex may improve detection of analytes by allowing the analyte to interact with separate complexes or molecules that may generate multiple copies of molecules that can ultimately disrupt the disruptable linker and release the signal-generating components (e.g., many or multiple copies of signal generating components). The presence of multiple copies of the components of signal-generating complexes, where each copy may be immobilized by separate disruptable (first component) or non-disruptable (second component) linkers, can enable a single molecule of analyte to create a chain reaction through one or more signal amplification complexes, thereby resulting in the generation of multiple copies of signal-generating complexes and a larger signal. The signal amplification complexes may comprise a catalytic agent that is immobilized to the solid substrate via a second disruptable linker. The second disruptable linker that immobilizes the catalytic agent may be different from the disruptable linker that immobilizes the first component of the signal-generating complex. The second disruptable linker may be responsive to the presence of the analyte and may be disrupted or cleaved in the presence of the analyte, thereby releasing a catalytic agent. The disruptable linker that immobilizes the first component of the signal-generating complex may be disruptable (e.g., cleavable) by the catalytic agent. By having multiple first components immobilized via multiple disruptable linkers, the catalytic agent may disrupt multiple disruptable linkers resulting in the release of multiple first components of signal-generating complexes and may generate multiple functional signal-generating complexes.

The signal amplification complexes may comprise an analyte binding module. The analyte binding module may bind to an analyte and result in the cleaving of second disruptable linker. For example, the analyte binding module may be an oligomer aptamer. The oligomer aptamer may comprise RNA, DNA, or a combination thereof. The oligomer aptamer may bind to a polypeptide analyte and may result in a cleavage of the linker that is dependent on the binding of the polypeptide target to the aptamer.

As described elsewhere herein, signal amplification complexes may comprise catalytic agents. Catalytic agents may comprise polypeptides (e.g., enzymes), DNA, RNA, or any other catalytic entities. For example, the catalytic agent may comprise a restriction enzyme. For example, the catalytic agent may be a DNase or RNase. In another example, a catalytic agent may be a nucleic acid and may trigger a strand displacement reaction. The nucleic acid may also function as an enzyme (e.g., DNAzyme or ribozyme). For example, the catalytic agent may be a 10-23 DNAzyme.

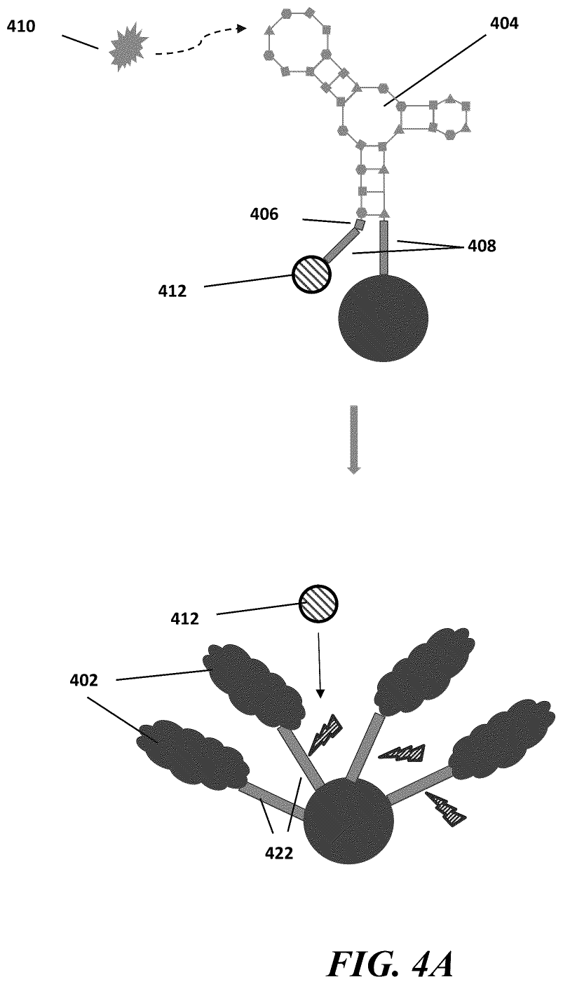

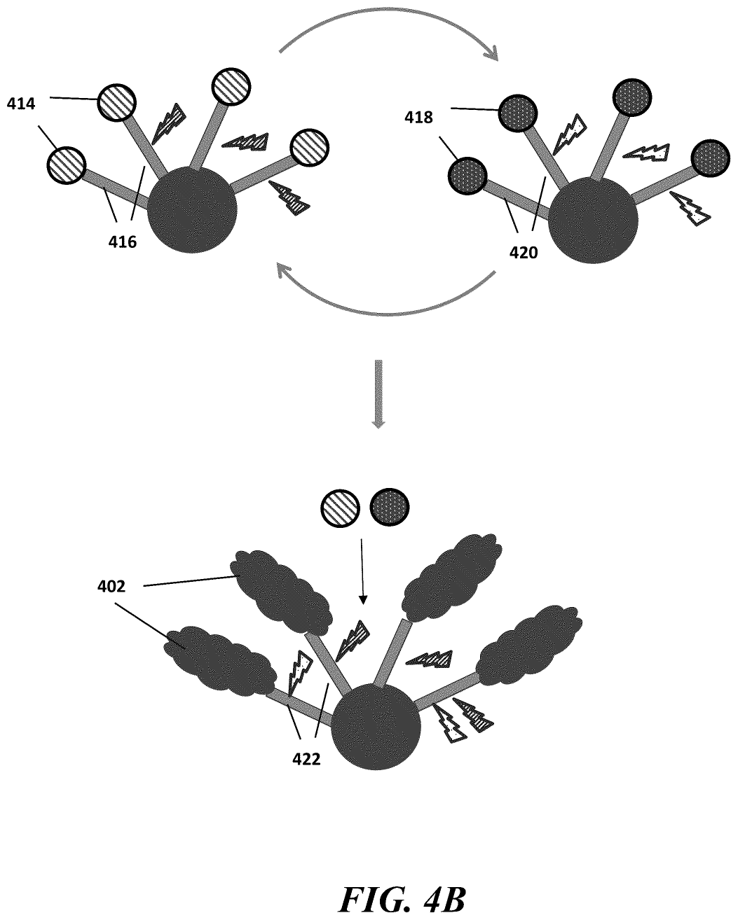

FIG. 4A demonstrates an example schematic of complexes for signal amplification. As shown, a catalytic agent 412 is attached to a linker 408 that comprises oligonucleotide aptamer 404 comprising a cleavable base 406. Aptamer 404 is able to bind to an analyte 410. Upon binding to the analyte, the aptamer can cleave the cleavable base 406 and release the catalytic agent 412. The catalytic agent may then be released into the solution. At a separate location, a plurality of first components 402 of the signal-generating complex are immobilized via a plurality of linkers 422. The released catalytic agent 412 may then cleave the linkers 422 and release one or more first components of the signal-generating complex. Each released first component of the signal-generating complex may then form functional signal-generating complexes with second components (not shown in FIG. 4A) and generate a signal that may be detected.

In some cases, there may be more than one signal-amplifying complex, which may allow for further degrees of signal amplification. For example, there may be a first signal-amplifying complex that comprises a first catalytic agent, and a second signal-amplifying complex that comprises a second catalytic agent. The catalytic agents may be used to generate an amplified signal in a variety of different reaction schemes. For example, a solid surface may comprise at least a first signal-amplifying complex that comprises a first catalytic agent, and a second signal-amplifying complex that comprises a second catalytic agent. The first catalytic agent may be released (e.g., via the interaction with an analyte), which may thereby release one or more copies of the second catalytic agent, (which may each thereby release one or more copies of a third catalytic agent, fourth catalytic agent, or an nth catalytic agent,) which thereby allows one or more copies of the first signal-generating components to be released. As a single copy of a first catalytic agent may be able to release multiple copies of the second catalytic agent, the resulting signal can be amplified with the presence of each additional signal amplifying complex.

FIG. 4B shows another schematic for a system with more than one signal amplification complex. As shown, there may be (i) a first signal-amplifying complex that comprises a plurality of first disruptable linkers 416 and a plurality of first catalytic agents 414 and (ii) a second signal-amplifying complex that comprises a plurality of second disruptable linkers 420, and a second catalytic agent 418. The second catalytic agent may be able to cleave the first disruptable linker and the first catalytic agent may be able to cleave a second disruptable linker. Either or both of the signal-amplifying complexes may comprise an analyte binding domain that results in an initial cleavage event. Alternatively, none of the two signal-amplifying complexes may comprise an analyte binding domain, and a third complex (e.g., another signal-amplifying complex) may comprise an analyte responsive module or mechanism (e.g., an analyte binding domain) that releases an initial catalytic agent. As such, the binding of the analyte to either complex or to a third complex may release one of the catalytic agents and in turn release the other of the catalytic agents. This may allow for a feedback loop in which the first catalytic agent and second catalytic agent are released in an exponential fashion. As each catalytic agent may release multiple copies of the other catalytic agent, the number of catalytic agents in solution can increase significantly, with additional catalytic agents being released with subsequent feedback loops and downstream reactions. At another location, a plurality of first components 402 of the signal-generating complex may be immobilized with a plurality of linkers 422 that can be disrupted by either catalytic agent, where each first component may be immobilized by a separate disruptable linker. Upon exposure to either or both of the catalytic agents, one or more first components of the signal-generating complexes can be released. Each first component of the signal-generating complex may then form functional signal-generating complexes with second components (not shown in FIG. 4B) and generate a signal that may be detected.

In various assays, the use of signal amplification complexes, the number of signal amplification complexes, their underlying interactions, and their use may be modulated or tuned. For example, as described above, the use of an assay with a linear correlation of analyte to signal complex may result in difficulty in detection of analytes at low concentration. However, the use of the linear correlation may be advantageous for quantification of an analyte, and the assay may be designed such to limit the number of signal amplification complexes. In cases where the analyte is present in low amounts, the use of signal amplification may be advantageous to create a larger signal from a low amount of analyte. For assays that may need more sensitivity, multiple (e.g., cross-interacting) signal amplification complexes may be used to generate a signal (e.g., in an exponential or polynomial amplification of the signal). The use of multiple signal amplification complexes may allow for the quantity of analyte to generally decouple from the intensity signal generated, whereby the presence of the analyte is expected to generate a signal above a threshold. In some cases, in highly amplified implementations, a number of microwells may be used to quantify the same analyte, wherein the number of microwells with a signal above a threshold (e.g., a binary threshold) may correlate with the concentration of that analyte.

### Disrupting Agents

As described elsewhere herein, the method and compositions may comprise a disruptable linker (e.g., a disruptable linker immobilizing a first component of a signal-generating component, a second disruptable linker that immobilizes a catalytic agent in a signal-amplifying complex). A disrupting agent may utilize a variety of different mechanisms to disrupt the linker. For example, disrupting agent may be a cleaving agent and may cleave or break covalent bonds. In another example, the disrupting agent may cause a non-covalent interaction to be disrupted, thereby disrupting the disruptable linker. For example, a disrupting agent may displace a component of the disruptable linker and cause disassociation. This disruption may be based on exchanging the non-covalent interaction(s) that hold the linker together with another non-covalent interaction(s) that does not maintain the linker's structure. For example, a linker may comprise an immobilized first member and a second member that interacts with the immobilized first member. The addition of a molecule of a non-immobilized first member may result in an exchange of interactions, in which the second member interacts with the non-immobilized member. This exchange of interactions can disrupt the linker structure and the second member would no longer be immobilized via its interaction with the immobilized first member. Similarly, the addition of another copy of the second member could cause the immobilized first member to exchange interactions, and form interactions with the second copy of the second member, thereby disrupting the linker. The disruption may be based at least on the disrupting agent making more favorable interactions with a member of the non-covalent interaction (e.g., have better binding kinetics or may be more thermodynamically stable) as compared to an initial member of the non-covalent interactions. For example, a disruptable linker may be composed of a nucleic acid duplex comprising a first strand and a second strand. A disrupting agent comprising a nucleic acid may disrupt the nucleic acid duplex by displacing one of the strands of nucleic acids (e.g., the first strand or second strand). The disrupting agent may comprise a different sequence, bases, or nucleotides as compared to the nucleic acids of the first or second strand that may result in the formation of a more thermodynamically favorable (or more kinetically favorable) duplex when the disrupting agent is a member of the duplex.

Various cleaving agents may be used to cleave a disruptable linker, thereby releasing a component of the signal-generating complex to allow generation of a signal. The cleaving agents may be activated by, or may comprise, an analyte. As such, the cleavage of the cleavable linker may be dependent on the presence of the analyte.

The cleaving agent may comprise a nuclease. The nuclease may specifically cleave a type of nucleic acid (e.g., an RNA or a DNA), or may cleave at a type of nucleobase. The analyte may comprise a nuclease, and thus the presence of the nuclease in the sample solution may cause the cleavage of the nuclease cleavage site in the linker. For example, the nuclease may comprise a DNase. For example, the nuclease may comprise an RNase. The nuclease may comprise a DNA or RNA dependent nuclease. For example, the nuclease may be dependent on the presence of a DNA or RNA in solution. The nuclease may be dependent on a specific nucleic acid sequence, (e.g., the nuclease may be dependent on the presence of the analyte which has a specific nucleic acid sequence). In an example, the nuclease may comprise a CRISPR-Cas nuclease. The CRISPR-Cas nuclease may be inactive without the presence of a specific nucleic acid. CRISPR-Cas nuclease may comprise a guide RNA(gRNA), and a Cas nuclease protein. In the presence of a specific nucleic acid sequence that hybridizes to the gRNA, the Cas nuclease may be able to cleave nucleic acids. Although CRISPR-Cas nuclease may be used to cleave nucleic acids at specific sites in the hybridizing nucleic acid sequence, CRISPR-Cas may also have collateral cleavage reactions that may indiscriminately cleave nucleic acids. A disruptable linker comprising nucleic acids may thus be cleaved using a CRISPR-Cas nuclease. The Cas nuclease may comprise a Cas3, Cas9, Cas12 (e.g., Cas12a, Cas12b), or Cas13 (e.g., Cas13a, Cas13b, Cas13d) or a Cas14 nuclease.

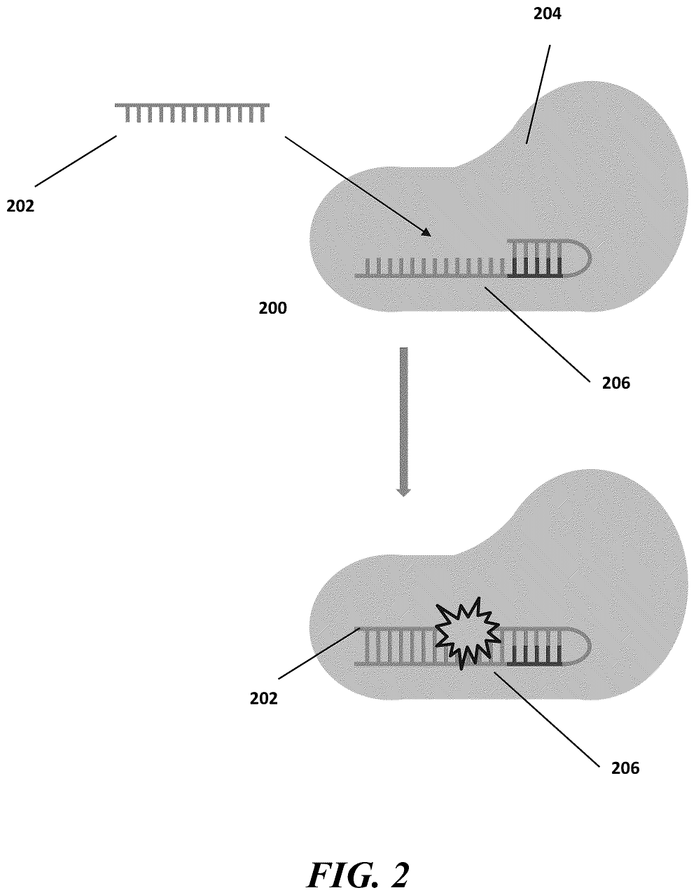

FIG. 2 demonstrates an example cleaving agent CRISPR-Cas, in which the cleaving activity can be analyte dependent. A CRISPR-Cas complex 200 may be generated by a Cas nuclease 204 and guide RNA (gRNA) 206. The gRNA may be configured or designed to hybridize or bind to a specific nucleic acid sequence. When an analyte comprising the specific nucleic acids sequence, shown as 202, is present, the CRISPR-Cas complex is able to hybridize to the analyte. As shown in the bottom schematic, the hybridization of the analyte to the gRNA may activate the CRISPR-Cas nuclease such that it may cleave nucleic acids (e.g., via collateral cleavage). In some embodiments, an additional Cas nuclease may be activated by the cleavage products of a different Cas nuclease. For example, Csm6 or Csx1 may be used to enhance performance of a Cas13 assay; once the Cas13 is activated, it cleaves a secondary construct, which in turn activates nucleases that perform cleavage. The CRISPR-Cas nuclease may cleave nucleic acid linkers (e.g., disruptable linkers) and release the component of the signal-generating complex.

The CRISPR Cas nuclease may be specific to a type of nucleic acid and may cleave a specific type of nucleic acid. For example, the Cas nuclease may comprise a Cas13 nuclease and hybridize to RNA specific targets. Upon activation the Cas nuclease may cleave an RNA target. The Cas nuclease may specifically cleave RNA and not DNA. This may be used advantageously to cleave a linker comprising RNA, while leaving intact those linkers that do not comprise RNA. For example, a first component of a signal-generating complex may be immobilized by a linker comprising RNA, and a second component of signal-generating complex may comprise a second linker that does not comprise RNA. Upon activation of the CRISPR-Cas nuclease complex, the nuclease may release the first component, while leaving the second component immobilized to the solid substrate.

Similarly, upon activation the Cas nuclease may cleave a DNA target. For example, the Cas nuclease may comprise a Cas12 nuclease and hybridize to DNA specific targets. The Cas nuclease may specifically cleave DNA and not RNA. This may be used advantageously to cleave a linker comprising DNA, while leaving intact those linkers that do not comprise RNA. For example, a first component of a signal-generating complex may be immobilized by a linker comprising DNA, and a second component of signal-generating complex may comprise a second linker that does not comprise DNA. Upon activation of the CRISPR-Cas nuclease complex, the nuclease may release the first component, while leaving the second component immobilized to the solid substrate.

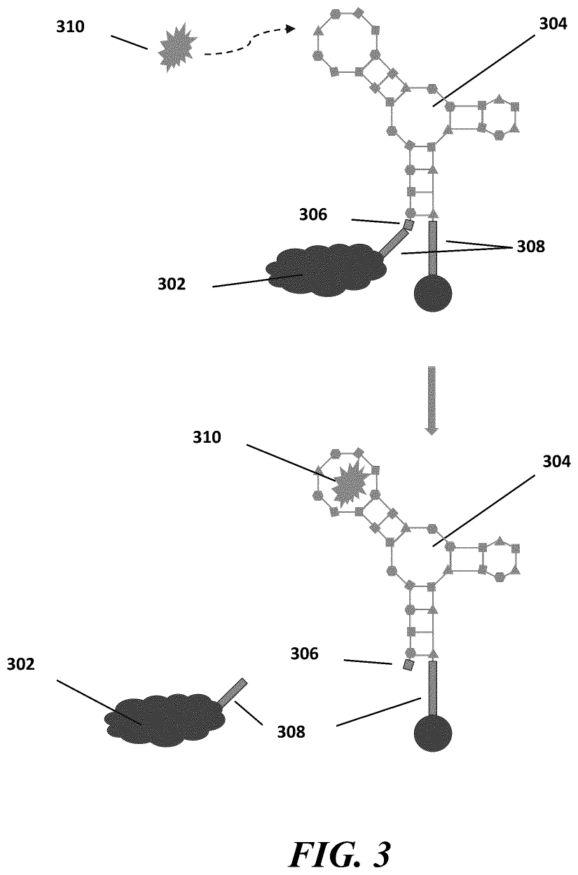

The cleavage agent may comprise a self-cleaving nucleic acid molecule. As described elsewhere in the present disclosure, the linker may comprise an oligonucleotide aptamer. The oligonucleotide aptamer may cleave itself at a nucleobase upon binding of an analyte to the aptamer. FIG. 3 demonstrates an example schematic of a self-disruptable or self-cleavable aptamer. As shown, a first component 302 is attached to a linker 308 that comprises oligonucleotide aptamer 304 comprising a cleavable base 306. Aptamer 304 is able to bind to an analyte 310. Upon binding to the analyte, the aptamer can cleave the cleavable base 306 and release the first component 302. The first component of the signal-generating complex may then form a functional signal-generating complex with a second component (not shown in FIG. 3) and generate a signal that may be detected.

As an example, the aptamer 304 may comprise an aptamer, as described by, for example, Ali et al., “A DNAzyme-Based Colorimetric Paper Sensor for Helicobacter pylori”, Angew Chem Int Ed Engl, 2019 Jul. 15; 58(29):9907-9911, which is incorporated by reference herein in its entirety. For example, an aptamer comprising the following Sequence BDHp3T4 from Table S2 of Ali et al. may be used: 5′ attachment-ATGCCATCGATGGTCTTTGGTATGTGGGGTCCGAGGGTAGAGCTCTGAACTCGTTTT TTTTTT-3′ attachment.

The linkers may comprise coupling moieties that allow the linkers to couple to other elements or molecules (e.g., the solid substrate, catalytic agents, etc.). For example, for an aptamer, such as one described above, the 5′ attachment point may show the point of connection to a first component of a signal-generation component (e.g., apoHRP), and the 3′ attachment point may show the point of connection to an immobilization moiety. Similarly, the 5′ and 3′ attachment point may be used for attachment to or immobilization of the second component of a signal-generating complex (e.g., hemin), or for attachment to or immobilization of signal amplification complexes described throughout this disclosure. The coupling moieties may be used in conjunction with non-disruptable linkers or disruptable linkers. The coupling moiety may be a biotin and may allow binding to a streptavidin (or other avidin derivative) coated surface. For example, the 5′ attachment point may be attached to a first or second component of a signal-generating complex (e.g., urease, apoperoxidase, or a first or second component of a signal-generating complex described herein).

The cleavage agent may comprise a protease. For example, the protease may comprise a serine protease, a threonine protease, a cysteine protease, aspartic protease, a glutamic protease, a metalloprotease, or other proteolytic enzyme. The protease may cleave a protease cleavage site. For example, the linker may comprise a chain of polypeptides that may comprise a known protease cleavage site. The protease can then cleave the protease cleavage site in the linker.

The disrupting agent may be a nucleic acid. The disrupting agent may be a nucleic acid that may cleave a disruptable linker. For example, the disrupting agent may be an RNA-cleaving DNAzyme, and may cleave an RNA sequence or RNA base in the disruptable linker.

The disrupting agent may be a ribozyme. The disrupting agent may disrupt the disruptable linker via strand displacement. For example, the disruptable linker may comprise a double stranded nucleic acid or double stranded region. A disrupting agent may comprise a nucleic acid with a sequence that is complementary to the double stranded nucleic acid and may be able to anneal to a strand of the double stranded nucleic acid. The disrupting agent may, by annealing to a strand of the double stranded nucleic acid in the disruptable linker, disrupt the disruptable linker. The disrupting agent may be able to anneal to one of the strands at a higher efficiency or affinity (e.g., via thermodynamically or kinetically favorable interactions) as compared to the originally hybridized double stranded region. For example, the disrupting agent may comprise modified nucleic acids, or may comprise a higher percentage complementarity (e.g., fewer mismatches) as compared to the originally hybridized double stranded region. For example, a first strand of a nucleic acid may be coupled to the solid substrate, and a second complementary strand may be coupled to the first component of the signal-generating complex. The disrupting agent may: (i) anneal to the first strand and displace the second strand; or (ii) may anneal to the second strand and displace the first strand. The second strand, no longer being annealed to the first strand, can now freely diffuse away from the solid substrate along with the first component of the signal-generating complex, thereby forming a functional signal-generating complex. The second strand may diffuse away as a single-stranded entity or a double-stranded entity (e.g., a duplex with a disrupting strand).

### Linker

In various aspects, a linker is used to link various elements to the solid substrate. For example, a linker may be used to link the components of the signal-generating complex to a solid substrate. Linkers may also be used in signal amplification complexes to immobilize the catalytic agent to the solid substrate. The linker may comprise a disruptable linker. The term “disruptable linker” may refer to a linker that can be degraded, cleaved, or allowed to dissociate, under reaction conditions of the methods described herein. The disruptable linker may link the components of the signal-generating complex to a solid substrate under a first set of conditions. Under a second set of conditions, the disruptable linker may be disrupted causing the connection between the components of the signal-generating complex and the solid substrate to be severed, thereby freeing the components from the solid substrate. Similarly, a disruptable linker (e.g., a second disruptable linker) may link the catalytic agents of signal amplifying complexes to a solid substrate under a first set of conditions. Under a second set of conditions, the disruptable linker may be disrupted causing the connection between the catalytic agents and the solid substrate to be severed, thereby freeing the catalytic agents from the solid substrate.

The linker may comprise nucleic acids. For example, the linker may comprise a ribonucleic acid (RNA). The linker may comprise a deoxyribonucleic acid (DNA). The linker may be disrupted via degradation of the nucleic acids. For example, the nucleic acids may be cleaved by a nuclease. The linker may comprise a nucleic acid duplex. For example, a duplex may be formed via the hydrogen bonding of two single strands comprising complementary sequences. A duplex may be disrupted resulting in two single strands of nucleic acids. A disruptable linker may comprise a duplex that is disrupted in the presence of an analyte. For example, an analyte may comprise a nucleic acid and may directly compete with a nucleic strand of the duplex (e.g., the analyte may comprise a same or substantially same sequence as a sequence in the duplex of the linker). In another example, the analyte may comprise a polypeptide. The linker may comprise a duplex comprising at least a single stranded element that comprises an aptamer that binds to the analyte. Upon binding of the analyte to the aptamer, the aptamer may trigger a conformational change in the single stranded element and disrupts the hydrogen bonds of the duplex. The linker may comprise specific sequences. The linker may comprise restriction enzyme recognition sites. For example, the linker may comprise a site that is recognized by XbaI and is cleaved by XbaI. The linker may comprise more than one restriction enzyme site and may be able to be cleaved by different restriction enzymes. For example, a linker may comprise an XbaI site and an AscI site, and may be cleaved by XbaI or AscI.

The linker may comprise a polypeptide or peptide bonds. For example, the linker may comprise a protein (e.g., a globular protein) or an unstructured chain of peptides. The polypeptide may comprise a component of a disruptable linker. For example, a polypeptide linker may be disrupted via degradation or cleavage using a protease. Protease activation via an analyte may allow the disruptable linker to be cleaved in the presence of an analyte. For example, the linker may comprise a chain of polypeptides that may comprise a known protease cleavage site. The analyte may comprise a protease, and thus the presence of the protease in the sample solution may cause the cleavage of the protease cleavage site in the linker.

In various aspects, the disruptable linker comprises a substrate for a catabolic enzyme. For example, as described elsewhere in the present disclosure, the disruptable linker may comprise nucleic acids that are degradable via a nuclease, or proteins that are degradable via a protease. Disruptable linkers may also comprise other substrates that may be degraded via an enzyme. For example, the linker may comprise a hyaluronic acid, which can be degraded by a hyaluronidase. An analyte may comprise hyaluronidase, and the use of a linker comprising hyaluronic acid may allow for the hyaluronidase (and its respective activity) to be detected by methods of the present disclosure.

The linker may comprise a stable or non-degradable linker. The stable or non-degradable linker may maintain the connection between the components of the signal generation complex and the solid substrate. The stable connection may prevent the signal-generating complex, or components thereof, from leaving the reaction vessel. This may allow the signal-generating complex to be confined into a reaction vessel and generate a signal in the reaction vessel. The stable or non-degradable linker may remain stable under reaction conditions or in the presence of the analyte.

Components of the signal-generating complex may be connected to the solid substrate by disruptable linkers or stable linkers. For example, a first component of a signal-generating complex may be attached via a disruptable linker, and a second component of a signal-generating complex may be attached via a stable linker. As the components are immobilized via linkers and are located in disparate locations of the solid substrate, the linkers prevent the components from interacting and forming a functional signal-generating complex. Upon addition of a stimulus, the disruptable liker may be disrupted releasing the first component of the signal-generating complex into solution. The second component attached via a stable linker may remain attached to the solid substrate while the first component is released into solution. The first component and second component can now interact, and the first component and second component may interact and remain bound to the solid substrate via the stable linker.

The disruptable linker may comprise a cleavable linker. A cleavable linker may be degraded via breaking of a covalent bond. For example, a cleavable linker may be susceptible to hydrolysis reaction which can cleave an oxygen-carbon bond of the cleavable linker. Other examples of a cleavable linker may comprise nitrogen-oxygen bonds, nitrogen-carbon bonds, phosphorous-oxygen bonds, sulfur-oxygen bonds, or sulfur-sulfur bonds. A cleavable bond may be cleaved by a cleavage agent, as described elsewhere in the present disclosure.

The cleavable linker may comprise a self-cleaving linker. For example, the linker may comprise a catalytic component that, when activated, cleaves the linker. The self-cleaving linker may comprise an oligonucleotide aptamer. The oligonucleotide aptamer may bind to the analyte and may induce cleavage of the linker. The oligonucleotide linker may comprise DNA. The oligonucleotide linker may comprise RNA. The oligonucleotide linker may comprise DNA and RNA.

A disruptable linker may comprise a dissociable linker. The dissociable linker may be disrupted via dissociation of two dissociable elements that are initially bound together. The two dissociable elements in a dissociable linker may be non-covalently attached to each other. For example, the elements may be attached via hydrogen bonding, pi-stacking, ionic bonding, Van der Waals interaction, or a non-polar interaction. For example, the dissociable linker may comprise a duplex of nucleic acids which are held together via hydrogen bonds. The duplex may be broken via the breaking of the hydrogen bonds (or pi-stacking interactions) resulting in separate single strands of nucleic acids. The dissociable linker may comprise a binding partner pair. A dissociable linker may comprise a molecule and an associated binding protein. For example, the dissociable linker may comprise a modified hyaluronic acid and an immobilized hyaluronic acid binding protein. In some embodiments, the modified hyaluronic acid comprises modifications that increase an ability of the modified hyaluronic acid to be displaced by unmodified hyaluronic acid. Free hyaluronic acid (or free hyaluronic acid binding protein) may be added to the solution and compete for the binding partner present in the linker. The interaction of binding partner pair may be disrupted by the free hyaluronic acid (or hyaluronic acid binding protein), thereby disrupting the linker.

The disruptable linker may comprise one or more disruptable regions. As described elsewhere herein, the disruptable linker may comprise different nucleotides (e.g., RNA, DNA), polypeptides, self-cleaving aptamers, or combinations thereof. The disruptable linker may comprise multiple different regions that are able to be disrupted independently. In this way, the disruptable linker may be able to be disrupted via more than one way. As described elsewhere herein, catalytic agents from signal-amplifying complexes may be used to cleave disruptable linkers. In some cases, where there is more than one type of signal-amplifying complex with more than one type of catalytic agent, the disruptable linker can be cleaved by any or multiple catalytic agents. This may allow for improved signal generation. For example, a linker may comprise RNA and DNA. The linker may be cleaved by an RNase and may also be cleaved by a DNase. The one or more disruptable regions may comprise a first disruptable region capable of being cleaved by a sequence-specific enzyme (e.g., a restriction enzyme) and a second disruptable region capable of being cleaved by a nucleotide-specific enzyme (e.g., DNase vs. RNase). For example, a disruptable linker may comprise (i) a first section of double stranded DNA that comprises sequences that are recognized by a restriction enzyme and (ii) a second section of RNA that can be cleaved by an RNase.

FIG. 4B (bottom) shows a construct comprising a first component of a signal-generating complex with disruptable linkers with multiple disruptable regions. Reactions with either catalytic agent 414 or catalytic agent 418 may release the first component of the signal-generating complex. Linker 424, for example, is shown to be able to react with both catalytic agent 414 and catalytic agent 418. In this way, there may be an increased likelihood of the first component of the signal-generating complex being released in response to an analyte in the sample.

### Biological Samples

The biological sample may be derived from or contain a tissue. The biological sample may be derived from or contain a biological fluid. For example, the biological sample may comprise a plasma sample, a serum sample, a buffy coat sample, a peripheral blood mononuclear cell (PBMC) sample, a red blood cell sample, a urine sample, a saliva sample, or other body fluid sample. The biological sample may comprise or be a pleural fluid sample, peritoneal fluid sample, amniotic fluid sample, cerebrospinal fluid sample, lymphatic fluid sample, sweat sample, tear sample, semen sample, mucus sample, or any combination of biological fluid. The biological sample may be derived from or contain an excretion from a subject. The bodily sample may comprise or be derived from fecal matter or a stool sample.

The biological sample may comprise nucleic acids. The biological sample may comprise or be a deoxyribonucleic acid DNA sample or a cell-free ribonucleic acid (RNA) sample. The nucleic acid may comprise a DNA (e.g., double-stranded DNA, single-stranded DNA, single-stranded DNA hairpins, cDNA, genomic DNA, germline DNA, circulating tumor DNA (ctDNA), cell-free DNA (cfDNA)), an RNA (e.g., cfRNA, mRNA, cRNA, miRNA, siRNA, miRNA, snoRNA, piRNA, tiRNA, snRNA), or a DNA/RNA hybrid. In some cases, the samples may comprise RNA and DNA. For example, a sample may comprise cfDNA and cfRNA, and the cfDNA and cfRNA may be analyzed by methods as described elsewhere herein.

The biological samples may be subjected to additional reactions or conditions prior to assaying. For example, the biological sample may be subjected to conditions that are sufficient to isolate, enrich, or extract nucleic acids, such as DNA molecules or RNA molecules. In another example, the biological sample may be subjected to conditions that are sufficient to isolate, enrich, or extract polypeptides, such as globular proteins.

The methods disclosed herein may comprise extraction reactions on one or more nucleic acids, polypeptides (e.g., proteins), or saccharides (e.g., polysaccharides) in a biological sample. The extraction reactions may lyse cells or disrupt nucleic acid interactions with the cell or with cellular proteins, such that the nucleic acids, polypeptides (e.g., proteins), or saccharides (e.g., polysaccharides) or may be isolated, purified, enriched, or subjected to other reactions. Similarly, extraction reactions may be performed that remove nucleic acids or lipids. For example, reactions may comprise the use of DNase or RNase to degrade nucleic acids.

In some cases, the biological sample may comprise multiple components. For example, the biological sample may comprise a whole blood sample. The biological sample may be subjected to reactions such to separate or fractionate a biological sample. For example, a whole blood sample may be fractionated, and cell free nucleic acids may be obtained. The whole blood sample may be fractionated using centrifugation or filtration (e.g., fibrous filter) such that blood cells may be separated from the plasma (which may contain cell free nucleic acid). A sample may be subjected to multiple rounds of separation or fractionation. Similarly, a cell sample may be lysed and subjected to centrifugation or filtration to remove cellular components.

The biological sample may be obtained or derived from a subject at a variety of times. The biological sample may be collected over 1, 2, 3, 4, 5, 6, 7, 8, 9, 10, 11, 12, 15, 20, 30, 40, 50, 60, 70, 80, 90, 100, 200, 300, 400, 500, 600, 700, 800, 900, or 1000 or more time points. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, or 60 or more hour period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, or 60 or more day period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, or 60 or more week period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, or 60 or more month period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, or 60 or more year period.

The samples may be derived from a variety of subjects. The subject may be a human subject. The subject may be suspected of having or may have a disorder or a disease. For example, the subject may be suspected of having a cancer. The subject may be suspected of having a specific cancer. The subject may be assessing various markers of general wellness. The subject may be assessing an optimal treatment for their disorder or disease based on their biomarker profile. For example, the subject may show evidence of a genetic mutation in a given sample which is successfully acted upon by a particular targeted therapy, or may express a gene signature in a particular sample that is indicative of better response to a particular kind of treatment.

### Analytes

The methods and systems described herein may be used to detect the presence of various analytes. The analyte may comprise a nucleic acid. The analyte may comprise a DNA (e.g., double-stranded DNA, single-stranded DNA, single-stranded DNA hairpins, cDNA, genomic DNA, germline DNA, circulating tumor DNA (ctDNA), cell-free DNA (cfDNA)), an RNA (e.g., cfRNA, mRNA, cRNA, miRNA, siRNA, miRNA, snoRNA, piRNA, tiRNA, snRNA), or a DNA/RNA hybrid. The analyte may be a nucleic acid of a particular or specific sequence. For example, the analyte may comprise a sequence of a gene or may comprise a gene mutation. For example, the analyte may comprise a DNA or mRNA (or a portion of the DNA or mRNA) corresponding to a gene. For example, the analyte may comprise a DNA or mRNA (or a portion of the DNA or mRNA) corresponding to a gene mutation. The detection of the analyte (e.g., DNA, mRNA) may be used to detect the expression of a gene or gene mutation in a sample or subject.

The analyte may comprise a polypeptide. For example, the analyte may comprise a protein receptor, an enzyme, glycoprotein, an antibody, an antibody fragment, a protein ligand, or other classes of proteins. The analyte may comprise a catabolic enzyme. For example, the analyte may cause a covalent bond breakage in a substrate or degrade a molecule. As described elsewhere in the present disclosure, a linker may comprise a substrate of a catabolic enzyme. In the presence, of the catabolic enzyme, the linker may be degraded and allow for the formation of the signal-generating complex. In this manner, a catabolic enzyme can be detected. For example, the catabolic enzyme may comprise hyaluronidase which can cleave a linker comprising hyaluronic acid.

The analytes may be associated with a condition of a subject from which a sample is derived from. For example, biomarkers may be associated with the presence of a disease or disorder. The presence of the biomarker (e.g., analyte) in a sample may be indicative of a disease or disorder in a subject. For example, the presence of a gene, or expression of a polypeptide may be indicative of a disease. For example, NMP22 may be used as a tumor marker for bladder cancer. The presence of UCAI RNA or NMP22 protein in a sample may be indicative of bladder cancer in a subject. As such, the method, systems, and compositions may be used to identify the presence of an analyte, and may indicate the presence of a disorder or disease in a subject.

### Signal Generation

In various aspects of the present disclosure, signals are generated to indicate the presence of an analyte. Signals may be generated via the signal-generating complexes described throughout the present disclosure. The signal-generating complex may use a pro-signal molecule to generate a signal. For example, the signal-generating complex may comprise an enzyme that on its own does not produce a detectable signal. The functional signal-generating complex may use a substrate to generate the signal. An initial substrate may comprise a pro-signal molecule that comprises a set of characteristics. For example, the pro-signal molecule may be colorless or non-luminescent. The pro-signal molecule may emit or absorb a set of wavelengths that may be used to detect the pro-signal molecule as present in a solution. This set of characteristics may be used as a baseline or reference value for the pro-signal molecule, and may be used to detect that the pro-signal molecule is unreacted. The functional signal-generating complex may react with or incorporate the pro-signal molecules such the characteristics of the resulting molecule(s) are different from the pro-signal molecule. For example, the pro-signal molecule may be colorless and upon reacting with the signal-generating complex may result in a molecule that has a color. The observation of the color may indicate reaction of the pro-signal molecule. In another example, the pro-signal molecule may be non-luminescent and upon reacting with the signal-generating complex may result in a molecule that is luminescent. The observation of the luminescence may indicate reaction of the pro-signal molecule. The pro-signal molecule may comprise an ABTS (2,2′-azino-bis(3-ethylbenzothiazoline-6-sulfonic acid)), OPD (o-phenylenediamine dihydrochloride, TMB (3,3′,5,5′tetramethylbenzidine dihydrochloride), DAB (3,3′-diaminobenzidine tetrahydrochloride), AEC (3-amino-9-ethylcarbazole), luminol, ADHP (10-Acetyl-3,7-dihydroxyphenoxazine), homovanillic acid, or other chromogenic molecule, chemiluminescent, or fluorescent substrates. The pro-signal molecule may react with a peroxidase to generate a colorimetric, luminescent, fluorescent, or electrochemical signal.

The methods and systems described herein may comprise signals that are generated by the signal-generating complex. The signals may comprise any type of signal that can be detected by a variety of detectors or instruments. The signal may comprise a colorimetric signal, fluorometric signal, electrochemical signal, or luminescent signal. The signal may be characterized in terms of a wavelength or a color. The signal may be characterized by the emission, absorbance, or transmission of a wavelength or set of wavelengths. For example, a signal may comprise a blue, green, or red colorimetric signal. The colorimetric signal may be determined based at least in part on measuring the absorbance at a particular wavelength or range of wavelengths, for example by determining a distribution of absorbances at various wavelengths and/or by utilizing a band pass filter to determine the absorbance within a particular range of wavelengths. The signal may comprise or be characterized by an intensity or amplitude. The signal may comprise a higher intensity or amplitude when the analyte is in higher concentrations. The signal intensity may be independent, or substantially independent, of an analyte concentration. In some cases, the signal may decrease in intensity, or a signal may be initially present and may be quenched or become substantially undetectable. The loss of a signal or decrease of a signal may also be used to determine the presence of an analyte. For example, a signal may be quenched via the addition or the presence of a quenching reagent. In another example, the signal may be shifted to another wavelength, and the signal intensity may decrease at a first wavelength and increase at a second wavelength.

The signals may be compared or normalized against a reference signal or other expected signal. For example, the signals may be compared against a background or negative control. A signal (or lack thereof) of a pro-signal molecule may be compared against a signal generated from a signal molecule. For example, the signal may comprise a higher absorbance within a particular range of wavelengths than a reference signal, or background or noise signal, and indicate the presence of an analyte. For example, a standard curve of references may be used to estimate the percentage of possible signal-generating complexes that have been created, which may indicate the presence of an analyte or its concentration. The standard curve of references may include a negative control, a positive control, and an intermediate control (or more than one intermediate control) with varying degrees of signal.

As described throughout the present disclosure, components of signal-generating complexes and signal amplifying complexes may be allowed to diffuse into a solution after disruption of a linker. Generally, signals are generated via the formation of functional signal-generating complexes, and the presence of signals created by the signal generating complex can be used to determine the presence of an analyte. However, additional assays may be performed on the solution (e.g., supernatant solution) to detect the presence of free components in solution, which would be indicative of a disruption event. For example, the solution (or an aliquot or portion of the solution) may be removed from the reaction vessel and assayed. The solution may be subjected to an immunoassay (e.g., enzyme-linked immunosorbent assay), a sequencing assay (e.g., a next generation sequencing assay), other nucleic acid detection assay (e.g., PCR, qPCR, droplet PCR), or other analytic assay. For example, the disruptable linker may comprise a nucleic acid. The linker may be disrupted and the nucleic acid (or a portion of the nucleic acid) may diffuse freely in solution. A sequencing assay or PCR assay may be used to detect the nucleic acid in solution. In another example, a polypeptide (e.g., a first component of a signal generating complex or restriction enzyme) may immobilized via a linker, and the disruption of the linker may allow the polypeptide to diffuse in solution. The solution may be subjected to an immunoassay configured to detect the presence of the polypeptide in solution. In some cases, the detection of the presence of the released nucleic acid or polypeptide in the solution (e.g., supernatant solution) by these assays can indicate disruption of the linker due to the presence of an analyte, whereas detection of the absence of the released nucleic acid or polypeptide in the solution (e.g., supernatant solution) can indicate that the non-disrupted linker is immobilized to the vessel surface due to the absence of the analyte.

### Solid Substrates and Reaction Vessels

The methods and systems may comprise the use of various reaction vessels or reaction surfaces. For example, a reaction vessel may be a well, microwell, flow cell, or test tube. The methods may be performed in a polymerase chain reaction (PCR) tube, a microtube or microcentrifuge tube, a microwell, a test tube, or other container that may hold a solution. In an example, the methods may be performed in a microwell of a microwell plate. The components may be immobilized to the bottom of a microwell plate and the methods may be performed by adding a sample to the microwell. Upon addition of a pro-signal molecule and generation of a signal, the microwell plate may be read using a plate reader to detect the signal.

As described throughout the disclosure, various elements are immobilized to a solid substrate. The solid substrate may the surface of a reaction vessel or container. For example, the solid substrate may be a well, microwell, flow cell, or test tube. The solid substrate may be the bottom surface of a well or microwell. For example, the solid substrate may be directly coupled to a linker via covalent bond. In some cases, the solid substrate can be functionalized with amine or carboxyl groups and can be covalently joined with the linker (e.g., through EDC/NHS chemistry). The solid substrate may be directly coupled to a linker via a non-covalent interaction. For example, the solid substrate may comprise polypeptide (e.g., streptavidin) that has been adsorbed to the solid substrate and may interact with a component of the linker, for example, a biotin moiety. The solid substrate may be a bead. For example, the solid substrate may be a streptavidin bead. The beads may be placed in a reaction vessel.

In various cases, different components of the systems provided in this disclosure are located at different areas of a solid substrate. The different areas may be different areas on a same solid substrate surface. For example, a microwell surface may comprise a first component at a first location, and a second component at a second location. The different areas may be different areas on different solid substrate surfaces that are in a same reaction vessel. For example, the first component may be immobilized to a first bead and the second component may be immobilized to a second bead. In some implementations, the components in the two locations are separated only by distance. In other implementations, the components in the two locations are additionally separated by a size-selective barrier, such as cellulose or nylon membranes with a defined pore size.

As described throughout the disclosure, the methods of the disclosure may be performed in a single reaction vessel or along a single surface, and may be performed without the addition of additional reagents. The elements used in the methods (e.g., components of the signal-generating complex) may be present in the single reaction vessel or surface prior to addition of a sample. This may allow for easier or faster detection of analytes. For example, in a single reaction vessel, a first component of a signaling-generating complex (e.g., apo-HRP), a second component of a signal generating complex (e.g., hemin), and a pro-signal molecule (e.g., ABTS) may all be present. Although the first component of a signaling-generating complex, the second component of a signal generating complex, and the pro-signal molecule may all be present in a reaction vessel, a signal may not be generated, as the components of the signal-generating complex are not associated and thus a functional signal-generating complex is not yet formed. Once an analyte is added (e.g., via addition of a sample), the disruptable linker may be disrupted (e.g., via analyte dependent nuclease activity or direct disruption via the analyte) and can allow for the functional signal generating complex to form, and the newly formed functional signal generating complex can then process the pro-signal molecule to generate a signal. The configuration of the compositions, systems, and methods can improve detection parameters, for example, increase sensitivity, specificity, and may allow for more rapid detection and testing.

### Detectors

The methods and systems of the present disclosure may comprise detection of signals. A detector may be used to detect the signals generated. The detector may detect a colorimetric signal. The detector may detect a fluorescent signal. The detector may detect an electrochemical signal. The detector may detect a luminescent signal.

The detector may comprise an imaging device. For example, the detector may comprise a camera. The camera may comprise a digital camera or a film camera. The detector may comprise light sensitive or light reactive sensors. For example, the detector may comprise a charged coupled device (CCD) sensor or Complementary Metal-Oxide Semiconductor (CMOS) sensor. The detector may comprise a photographic film, and may comprise light sensitive structures (e.g., silver halide crystals). The detector may detect light and integrate the detected light into an image. The imaging device may comprise a component of a mobile device. For example, an assay may be run using the methods of the present disclosure, and a user may use a mobile device to generate an image of the output of the assay.

The detector may comprise a photometer or photodetector. For example, the detector may comprise a spectrophotometer, a fluorometer, or luminometer. For example, a detector may measure a wavelength (or set of wavelengths) that is emitted, absorbed, or transmitted (e.g., from a photon emitted from a luminescent or fluorescent molecule). For example, a monochromator may be used to detect light at a specific set of wavelengths. Optical filters may also be used to filter out specific wavelengths of light, such to allow for the detection of specific wavelengths. For example, a longpass or shortpass filter may be used. The detector (or associated elements) may emit a wavelength of light. For example, the detector may emit a wavelength of light that is absorbed by a fluorescent molecule. The fluorescent molecule may absorb a wavelength of light and emit a different wavelength of light that may be detected by the detector.

The detector may detect an electrochemical signal. For example, the detector may detect an electron transfer (e.g., redox reaction). The detector may comprise a potentiostat. For example, the detector may measure the potential or voltage difference between two electrodes. The electrodes may be placed in a sample solution and the voltage may be measured in the solution. An electrochemical signal (e.g., a redox reaction) may be detected using a potentiostat, using for example, cyclic voltammetry or other techniques to detect a change in potential.

### Detection

In various aspects, the systems and methods may comprise accurately quantifying the concentration of an analyte in a sample. For example, analyte levels measured with these methods or systems may be between 60-140%, 70-130%, 75-125%, 80-120%, 85-115% 90-110%, 95-105%, 98-102%, or 99-101% of the known analyte level (e.g., as measured or verified through recovery studies).

In various aspects, the systems and methods may comprise detection of a presence or absence of an analyte at a certain accuracy, sensitivity, or specificity, or combinations of a certain accuracy, sensitivity, or specificity. For example, the methods or systems may comprise detecting the presence or the absence of a concentration (e.g., a clinically relevant concentration) of an analyte in a sample at an accuracy of at least about 60%, at least about 70%, at least about 75%, at least about 80%, at least about 85%, at least about 90%, at least about 95%, at least about 98%, or at least about 99%. For example, the methods or systems may comprise detecting the presence or the absence of a concentration of an analyte in a sample at a sensitivity of at least about 60%, at least about 70%, at least about 75%, at least about 80%, at least about 85%, at least about 90%, at least about 95%, at least about 98%, or at least about 99%. For example, the methods or systems may comprise detecting the presence or the absence of a concentration of an analyte in a sample at a specificity of at least about 60%, at least about 70%, at least about 75%, at least about 80%, at least about 85%, at least about 90%, at least about 95%, at least about 98%, or at least about 99%. The systems and methods described herein may comprise detection of a presence or absence of a concentration of an analyte at a certain sensitivity and specificity, such as a sensitivity or specificity provided in this disclosure. For example, the methods or systems may comprise detecting the presence or the absence of a concentration of an analyte in a sample at a sensitivity of at least about 60% and a specificity of at least about 60%. For example, the methods or systems may comprise detecting the presence or the absence of a concentration of an analyte in a sample at a sensitivity of at least about 60% and a specificity of at least about 70%. For example, the methods or systems may comprise detecting the presence or the absence of a concentration of an analyte in a sample at a sensitivity of at least about 60% and a specificity of at least about 80%. For example, the methods or systems may comprise detecting the presence or the absence of a concentration of an analyte in a sample at a sensitivity of at least about 60% and a specificity of at least about 90%. For example, the methods or systems may comprise detecting the presence or the absence of a concentration of an analyte in a sample at a sensitivity of at least about 60% and a specificity of at least about 99%. For example, the methods or systems may comprise detecting the presence or the absence of a concentration of an analyte in a sample at a sensitivity of at least about 70% and a specificity of at least about 60%. For example, the methods or systems may comprise detecting the presence or the absence of a concentration of an analyte in a sample at a sensitivity of at least about 80% and a specificity of at least about 60%. For example, the methods or systems may comprise detecting the presence or the absence of a concentration of an analyte in a sample at a sensitivity of at least about 90% and a specificity of at least about 60%. For example, the methods or systems may comprise detecting the presence or the absence of a concentration of an analyte in a sample at a sensitivity of at least about 99% and a specificity of at least about 60%. For example, the methods or systems may comprise detecting the presence or the absence of a concentration of an analyte in a sample at a sensitivity of at least about 70% and a specificity of at least about 70%. For example, the methods or systems may comprise detecting the presence or the absence of a concentration of an analyte in a sample at a sensitivity of at least about 80% and a specificity of at least about 80%. For example, the methods or systems may comprise detecting the presence or the absence of a concentration of an analyte in a sample at a sensitivity of at least about 90% and a specificity of at least about 90%. For example, the methods or systems may comprise detecting the presence or the absence of a concentration of an analyte in a sample at a sensitivity of at least about 99% and a specificity of at least about 99%.

The methods and systems may allow for the detection of an analyte at above a concentration (e.g., a threshold concentration). For example, the methods and systems may indicate that an analyte is present at a concentration above a threshold concentration. The threshold concentration may be a concentration that is deemed or determined to be relevant to a biological activity or clinical parameter. The threshold concentration may be based at least in part on a reference sample or data. By detecting that an analyte is present above a certain concentration may allow for the detection to correlative or indicative of a disorder, disease, or other indication. For example, the methods detecting an analyte as present above a threshold concentration may indicate that the subject has a particular disease. The threshold concentration may be a clinically relevant concentration, such that the presence of the analyte above the clinically relevant concentration is indicative of a clinical parameter (e.g., a disease state). For example, the methods and systems may allow for detection of an analyte as present in a concentration of at least 10 picomolar (pM). For example, the methods and systems may allow for detection of an analyte as present in a concentration of at least 50 picomolar (pM), 100 pM, 200 pM, 300 pM, 400 pM, 500 pM, 600 pM, 700 pM, 800 pM, 900 pM, 1 nM, 10 nM, 100 nM, 1 μM or more. For example, the methods and systems may allow for detection of an analyte as present in a concentration of no more than 10 pM, 50 pM, 100 pM, 200 pM, 300 pM, 400 pM, 500 pM, 600 pM, 700 pM, 800 pM, 900 pM, 1 nM, 10 nM, 100 nM, 1 μM or less.

The method may be performed such that an analyte may be detected within a certain amount of time. For example, the methods may allow for rapid detection of analytes. The methods of the disclosure may be performed in no more than 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 35 minutes, 40 minutes, 45 minutes, 50 minutes, 55 minutes, 60 minutes, or less. The methods of the disclosure may be performed in no more than 1 hour, 2 hours, 3 hours, 4 hours, 5 hours, 6 hours, 7 hours, 8 hours, 9 hours, 10 hours, or less. The methods may be performed within a certain amount of time and may be able to detect an analyte (e.g., an analyte at a clinically relevant concentration) at a level of accuracy, sensitivity, and/or specificity. For example, the method may be performed in less than 30 minutes and can detect an analyte as present in at least a concentration (e.g., a clinically relevant concentration) at at least a 90% specificity and at least a 90% sensitivity.

In various aspects, the systems and methods may comprise detection of a presence or absence of a disease, disorder, or condition (e.g., cancer) at a certain accuracy, sensitivity, or specificity (or combinations of certain accuracy, sensitivity, or specificity), which may be based at least in part on detection of an analyte. For example, the methods or systems may comprise detecting the presence or the absence of a disease, disorder, or condition in the subject at an accuracy of at least about 60%, at least about 70%, at least about 75%, at least about 80%, at least about 85%, at least about 90%, at least about 95%, at least about 98%, or at least about 99%. The methods or systems may comprise detecting the presence or the absence of a disease, disorder, or condition in the subject at a sensitivity of at least about 60%, at least about 70%, at least about 75%, at least about 80%, at least about 85%, at least about 90%, at least about 95%, at least about 98%, or at least about 99%. The methods or systems may comprise detecting the presence or the absence of a disease, disorder, or condition in the subject at a specificity of at least about 60%, at least about 70%, at least about 75%, at least about 80%, at least about 85%, at least about 90%, at least about 95%, at least about 98%, or at least about 99%.

### Computer Systems

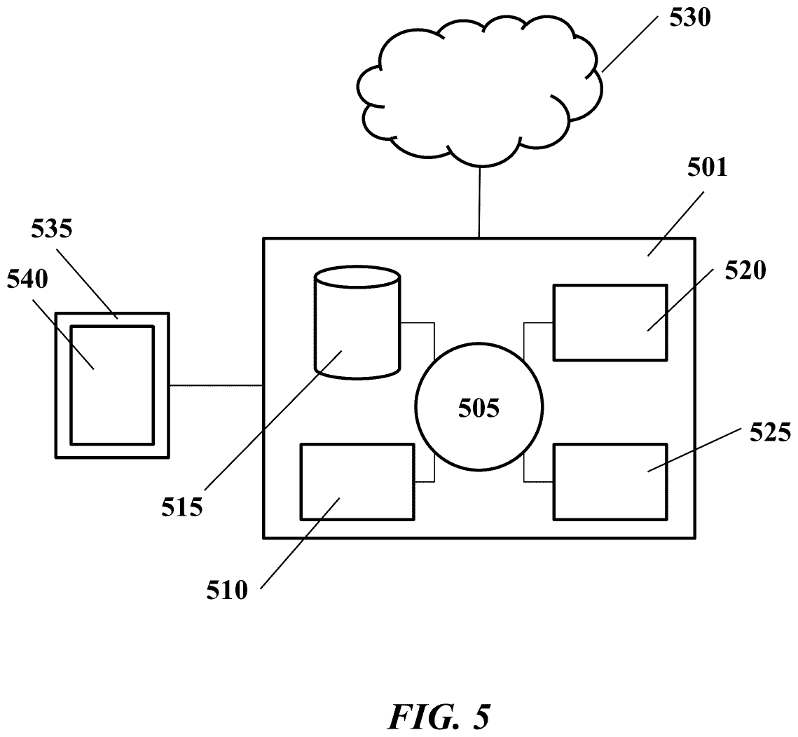

The present disclosure provides computer systems that are programmed to implement methods of the present disclosure. FIG. 5 shows a computer system 501 that is programmed or otherwise configured to perform analysis or operations of the methods, for example, generate or record an image, detect a color change, output a determination of the presence and/or quantity of an analyte. The computer system 501 can regulate various aspects of methods and systems of the present disclosure, such as, operate a detector, modulate an exposure time of an image or detect the presence of a color, for example. The computer system 501 can be an electronic device of a user or a computer system that is remotely located with respect to the electronic device. The electronic device can be a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 501 can communicate with a remote computer system of a user (e.g., a medical professional or patient). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine-readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, display a visual output relating to a detection of a color, and/or the presence of an analyte based at least in part on the detected color. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 505. The algorithm can, for example, identify an assay as generating a color and determining that a sample is a positive based at least in part on the generated color.

### EXAMPLES

### Example 1: Assay for Detection of an Analyte

A sample from an individual is obtained. A microwell plate comprising a microwell is prepared for a sample. The microwell comprises an apo-horseradish peroxidase that is immobilized to the microwell by a linker. This first linker comprises an immobilized aptamer sequence comprised of primarily DNA bases with an internal region of at least one RNA base. This aptamer is able to bind to NMP22, and this NMP22-bound aptamer sequence is able to cleave itself at the internal RNA region. At a second location, a modified hemin is immobilized to the microwell using a second linker. The second linker comprises a linear carbonaceous chain such as polyethylene glycol. The sample is added to the well. The sample comprises NMP22 protein which binds to the aptamer comprising the first linker. The bound aptamer then cleaves itself and releases the apo-horseradish peroxidase into the solution. The apo-horseradish peroxidase is able to interact with the immobilized modified hemin, and generates an active holo-horseradish peroxidase. TMB (3,3′,5,5′-tetramethylbenzidine) is added into the microwell, which is colorless. The holo-horseradish peroxidase is able to oxidize the TMB, which results in an oxidized TMB which comprises a color. Alternatively, TMB may be initially present in the microwell and may create a signal once the active holo-horseradish peroxidases are generated.

The microwell plate is then moved to a plate reader to read the wavelengths that are emitted, transmitted, or absorbed from the well. For example, the magnitude of absorbance at characteristic wavelengths of TMB (e.g., 650 nanometers (nm)) is correlated with the quantity of analyte in the sample, such that a strong absorbance is indicative that the analyte is present in the sample.

### Example 2: Assay for Detection of an Analyte

A sample from an individual is obtained. A microwell plate comprising a microwell is prepared for a sample. The microwell comprises an apo-horseradish peroxidase that is immobilized to the microwell by a first linker. This first linker comprises a polynucleotide. At a second location, a modified hemin is immobilized to the microwell using a second linker. The second linker comprises a linear carbonaceous chain such as polyethylene glycol. A cleaving agent CRISPR-Cas is present in which any cleaving activity is analyte dependent. A CRISPR-Cas complex comprises a Cas nuclease and guide RNA (gRNA). The gRNA may be configured or designed to hybridize or bind to a specific nucleic acid sequence of an analyte When an analyte comprising the specific nucleic acids sequence is present, the CRISPR-Cas complex is able to hybridize to the analyte. The hybridization of the analyte to the gRNA activates the CRISPR-Cas nuclease such that it may cleave nucleic acids (e.g., via collateral cleavage) The CRISPR-Cas nuclease cleaves nucleic acid linkers and releases the apo-horseradish peroxidase.

The sample is added to the well. The sample comprises the analyte which hybridizes to the gRNA. The activated CRISPR-Cas cleaves the first linker comprising a polynucleotide and releases the apo-horseradish peroxidase into the solution. The apo-horseradish peroxidase is able to interact with the immobilized modified hemin, and generates an active holo-horseradish peroxidase. TMB (3,3′,5,5′-tetramethylbenzidine) is added into the microwell, which is colorless. The holo-horseradish peroxidase is able to oxidize the TMB, which results in an oxidized TMB which comprises a color. Alternatively, TMB may be initially present in the microwell and may create a signal once the active holo-horseradish peroxidases are generated. The microwell plate is then moved to a plate reader to read the wavelengths that are emitted, transmitted, or absorbed from the well. For example, the magnitude of absorbance at characteristic wavelengths of TMB (e.g., 650 nanometers (nm)) is correlated with the quantity of analyte in the sample, such that a strong absorbance is indicative that the analyte is present in the sample.

### Example 3: Detection of an Analyte Using an Assay System With Signal-Amplifying Complexes

A sample from an individual is obtained. A microwell plate comprising a microwell is prepared for a sample. The assay is designed to detect RNase in the sample. The microwell comprises many copies of apo-horseradish peroxidase that are immobilized to the microwell by multiple linkers at a first location, where each copy is immobilized with a separate linker. These linkers comprise DNA that comprises a sequence that is recognized and cleaved by a XbaI enzyme (e.g., they are disruptable DNA linkers). At a second location, multiple modified hemin are immobilized to the microwell using a plurality of second linkers. Each second linker comprises a linear carbonaceous chain such as polyethylene glycol. At a third location, signal amplification complexes are present, with a given signal amplification complex comprising RNA, the natural substrate for the RNase, which is modified to have biotin on one end and XbaI on the other, and the biotin is immobilized to the solid substrate via interaction with streptavidin. As such, the XbaI is unable to interact with the first linker, and cleavage of this RNA linker releases the immobilized XbaI and allows it to be released and cleave the linker.

The sample is added to the well. The RNase cleaves the RNA in the signal amplification complexes, thereby releasing multiple copies of XbaI which are able to diffuse into the solution. The disruptable DNA linkers, which each contain an XbaI enzyme recognition site, are then cleaved, thereby releasing many apo-peroxidase molecules. The apo-horseradish peroxidases are able to interact with the immobilized modified hemins and generate multiple active holo-horseradish peroxidases.

Signals may then be generated from the active holo-horseradish peroxidases. In one version, TMB (3,3′,5,5′-tetramethylbenzidine) is added into the microwell, which is colorless. The holo-horseradish peroxidases are able to oxidize the TMB, which results in an oxidized TMB which comprises a color. Alternatively, TMB may be initially present in the microwell and may create a signal once the active holo-horseradish peroxidases are generated.

The microwell plate can then be read in order to determine the presence of an analyte. One way of reading the microwell plate is the use of an imaging device. A mobile device (e.g., cell phone) with a camera can be used to image the microwell plate. An application on the mobile device can be used detect the color and intensity of the microwells and can output a determination relating to the presence of the analyte.

Additionally, or alternatively, the microwell plate can be read using a plate reader. The microwell plate is moved to a plate reader to read the wavelengths that are emitted, transmitted, or absorbed from the well. For example, the magnitude of absorbance at characteristic wavelengths of TMB (e.g., 650 nm) is correlated with the quantity of analyte in the sample, such that a strong absorbance is indicative that the RNase is present in the sample.

Similar assays may be designed to detect RNA of a specific sequence by using a Cas complex (such as those described in this disclosure) that recognize specific RNA sequences and collaterally cleave RNA. The collateral cleavage may cleave the RNA substrate of the signal amplification complexes described in the example (as opposed to the RNase described above) and result in a signal via release of apoperoxidase molecules. Additionally, XbaI (and the accompanying XbaI restriction sites) described in this example can be interchanged for other restriction enzymes and their corresponding recognition sites.

### Example 4: Detection of an Analyte Using an Assay System With Multiple Signal-Amplifying Complexes

A sample from an individual is obtained. A microwell plate comprising a microwell is prepared for a sample. The assay is designed to detect RNase in the sample. The microwell comprises many copies of apo-horseradish peroxidase that are immobilized to the microwell by multiple linkers at a first location, where each copy is immobilized with a separate linker. These linkers comprise DNA that comprises a first sequence that is recognized and cleaved by an AscI enzyme and a second sequence that is recognized and cleaved by an XbaI. At a second location, multiple modified hemin are immobilized to the microwell using a plurality of second linkers. Each second linker comprises a linear carbonaceous chain such as polyethylene glycol. At a third location, a first type of signal amplification complex is present. The first type of signal amplification complex can comprise RNA, the natural substrate for the RNase, which is modified to have biotin on one end and an XbaI on the other, and the biotin is immobilized to the solid substrate via interaction with streptavidin. As such, the XbaI is unable to interact with the first linker, and cleavage of this RNA linker would release the immobilized XbaI and allow it to cleave the linker that immobilizes the apo-horseradish peroxidase. At a fourth location, a second type of signal amplification is present, comprising multiple AscI molecules that are immobilized by DNA linkers comprising an XbaI enzyme recognition site (e.g., each AscI molecule is immobilized by a DNA linker comprising a XbaI enzyme recognition site). This allows the release of one molecule of XbaI to release multiple molecules of AscI, in addition to the direct release of multiple molecules of apo-peroxidase by XbaI. AscI and XbaI may then diffuse to the region where apo-peroxidase is immobilized.

The sample is added to the well. The RNase cleaves the RNA in multiple copies of the first type of signal amplification complex, thereby releasing XbaI which is able to diffuse into the solution. The DNA linkers in both the second type of signal amplification complex which contain XbaI enzyme recognition sites, and those linkers immobilizing the apo-peroxidase molecules are then cleaved, releasing AscI and some apo-peroxidase molecules. The AscI is released and is able to cleave the linkers immobilizing the apo-peroxidase molecules and releases additional apoperoxidase molecules. The apo-horseradish peroxidases are able to interact with the immobilized modified hemins and generate multiple active holo-horseradish peroxidases.

Signals may then be generated from the active holo-horseradish peroxidases. In one version, TMB (3,3′,5,5′-tetramethylbenzidine) is added into the microwell, which is colorless. The holo-horseradish peroxidases are able to oxidize the TMB, which results in an oxidized TMB which comprises a color. Alternatively, TMB may be initially present in the microwell and may create a signal once the active holo-horseradish peroxidases are generated.

The microwell plate can then be read in order to determine the presence of an analyte. One way of reading the microwell plate is the use of an imaging device. A mobile device (e.g., cell phone) with a camera can be used to image the microwell plate. An application on the mobile device can be used detect the color and intensity of the microwells and can output a determination relating to the presence of the analyte.

Additionally, or alternatively, the microwell plate can be read using a plate reader. The microwell plate is moved to a plate reader to read the wavelengths that are emitted, transmitted, or absorbed from the well. For example, the magnitude of absorbance at characteristic wavelengths of TMB (e.g., 650 nm) is correlated with the quantity of analyte in the sample, such that a strong absorbance is indicative that the RNase is present in the sample.

Similar assays may be designed to detect RNA of a specific sequence by using a Cas complex (such as those described in this disclosure) that recognize specific RNA sequences and collaterally cleave RNA. The collateral cleavage may cleave the RNA substrate of the first type of signal amplification complex described in the example (as opposed to the RNase described above) and result in a signal via release of AscI and apoperoxidase molecules. Additionally, XbaI and AscI (and the accompanying XbaI and AscI restriction sites) described in this example can be interchanged for other restriction enzymes and their corresponding recognition sites.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of generating a signal indicative of a presence of an analyte, said method comprising:
(a) providing a solid substrate comprising:
(i) a first component of a signal-generating complex, wherein said first component is immobilized to said solid substrate via a disruptable linker at a first location of a reaction vessel, and
(ii) a second component of said signal-generating complex, wherein said second component is immobilized to said solid substrate at a second location of said reaction vessel;
(b) contacting a sample with said solid substrate, wherein said linker is disrupted based at least in part on a presence of said analyte, thereby releasing said first component; and
(c) associating said first component to said second component to form a functional signal-generating complex immobilized to said solid substrate, wherein said functional signal-generating complex generates a signal indicative of a presence of said analyte in said sample.

2. The method of claim 1, wherein said first component comprises an enzyme.

3. The method of claim 1, wherein said second component comprises a cofactor.

4. The method of claim 1, wherein said functional signal-generating complex comprises a holoenzyme.

5. The method of claim 1, further comprising, subsequent to (c), incorporating a pro-signal molecule into said functional signal-generating complex and processing said pro-signal molecule to generate said signal.

6. The method of claim 5, wherein, prior to said incorporating said pro signal molecule, detecting an additional signal, wherein said additional signal is non-fluorescent, non-luminescent, or colorless, or comprises an absorbance at one or more wavelengths below a threshold.

7. The method of claim 1, wherein said signal comprises a colorimetric, fluorometric, electrochemical, or luminescent signal.

8. The method of claim 1, wherein said disruptable linker comprises a ribonucleic acid (RNA), a deoxyribonucleic acid (DNA), a polypeptide, or a saccharide.

9. The method of claim 1, wherein said disruptable linker is cleaved by said analyte.

10. The method of claim 1, further comprising, prior to b), contacting said solid substrate with a disrupting agent.

11. The method of claim 10, wherein said disrupting agent disrupts non-covalent interactions in the disruptable linker.

12. The method of claim 10, wherein said disrupting agent comprises a CRISPR-enzyme complex.

13. The method of claim 12, wherein said CRISPR-enzyme complex comprises a guide RNA, wherein said guide RNA comprises a sequence that a complement or reverse complement of at least a portion of said analyte.

14. The method of claim 12, wherein said disruptable linker is cleaved by said CRISPR-enzyme complex via collateral cleavage activity that has been activated by the presence of said analyte.

15. The method of claim 1, wherein said disruptable linker comprises a self-cleaving linker.

16. The method of claim 15, wherein said self-cleaving linker comprises an oligonucleotide aptamer able to bind to said analyte, and wherein said self-cleavable linker is cleaved upon binding to said analyte.

17. The method of claim 1, wherein said solid substrate further comprises a signal-amplifying complex, wherein said signal-amplifying complex comprises a catalytic agent immobilized to said solid substrate via a second disruptable linker.

18. The method of claim 17, wherein said signal-amplifying complex further comprises an analyte binding module capable of binding to said analyte.

19. The method of claim 18, wherein said second disruptable linker is disrupted upon binding of said analyte to said analyte-binding module, thereby releasing said catalytic agent.

20. The method of claim 1, wherein said analyte comprises a ribonucleic acid (RNA), a deoxyribonucleic acid (DNA), a polypeptide, or a saccharide.

21. The method of claim 1, wherein said reaction vessel comprises a well.

22. The method of claim 1, further comprising, subsequent to (c), using a detector to detect said signal indicative of said presence or a quantity of said analyte in said sample.

23. A system for detecting an analyte in a sample, comprising:
a solid substrate comprising:
(i) a first component of a signal-generating complex, wherein said first component is immobilized to said solid substrate via a disruptable linker at a first location of a reaction vessel, and
(ii) a second component of said signal-generating complex, wherein said second component is immobilized to said solid substrate at a second location of said reaction vessel;
wherein said disruptable linker is cleaved based on a presence of said analyte, upon contacting a sample with said solid substrate thereby releasing said first component;
wherein a functional signal-generating complex is formed upon associating said first component to said second component, wherein a signal is generated from said functional signal-generating complex.
wherein said disruptable linker is cleaved based on a presence of said analyte, upon
contacting a sample with said solid substrate thereby releasing said first component; wherein a functional signal-generating complex is formed upon associating said first component to said second component, wherein a signal is generated from said functional signal-generating complex.
